(19) United States
(12) Patent Application Publication
(10) Pub. No.: US 2025/0352755 A1
(43) Pub. Date: Nov. 20, 2025

(54) RESPIRATORY SUPPORT DEVICE

(71) Applicant: Fisher & Paykel Healthcare Limited, Auckland (NZ)

(72) Inventors: Matthew Jon PAYTON, Auckland (NZ); Sam James TULLETT, Auckland (NZ); Taylor James EDWARDS, Auckland (NZ); David Petrus BRUWER, Auckland (NZ)

(21) Appl. No.: 18/872,196

(22) PCT Filed: Jun. 22, 2023
(86) PCT No.: PCT/IB2023/056441

§ 371 Date: Dec. 5, 2024

Related U.S. Application Data
(60) Provisional application No. 63/354,647, filed on Jun. 22, 2022.

(51) Int. Cl. A61M 16/18; A61M 16/00; A61M 16/04; A61M 16/06; A61M 16/20; A61M 16/22

(52) U.S. Cl. CPC A61M 16/18; A61M 16/0045; A61M 16/0605; A61M 16/209; A61M 16/22; A61M 2016/0027; A61M 2016/0036; A61M 16/0465; A61M 16/0666

(57) ABSTRACT

A device for providing respiratory support to a patient comprises a first gas flow path; a second gas flow path and a first gases port configured to receive gases from either of the first gas flow path and the second gas flow path. The device comprises a switching mechanism operable to switch flow to the first gases port between the first gas flow path and the second gas flow path. Gases from the first gases port are provided to the patient for respiratory support.

### TECHNICAL FIELD

The present disclosure relates to provision of respiratory support to a patient, and to devices and systems that provide for switching between modes of respiratory support provided to the patient. It relates particularly but not exclusively to devices for switching between modes of respiratory support which provide ease of use in the clinical setting.

### BACKGROUND OF INVENTION

Patients may lose respiratory function during anaesthesia, or sedation, or more generally during certain medical procedures. Prior to a medical procedure a patient may be pre-oxygenated by a medical professional to provide a reservoir of oxygen saturation. Pre-oxygenation and CO₂ flushing/washout may be carried out with high flow respiratory support via a nasal cannula or other patient interface.

High flow systems may be present in the operating theatre for use during anaesthetic or sedation procedures, or other medical procedures. High flow respiratory support has been found effective in meeting or exceeding the patient's normal inspiratory demand, to increase oxygenation of the patient, reduce the work of breathing or perform Transnasal Humidified Rapid-Insufflation Ventilatory Exchange (THRIVE). Pre-oxygenation using high flow systems prior to administration of anaesthesia or sedation provides an oxygen reservoir and extends safe apnoea time. Additionally, high gas flows may generate a flushing effect in the nasopharynx such that the anatomical dead space of the upper airways is flushed by the high incoming gas flows. This creates a reservoir of fresh gas available for each and every breath, while minimising re-breathing of carbon dioxide, nitrogen, etc. THRIVE is the provision of a high flow of respiratory gases to the patient when the patient is apnoeic, which occurs when the anaesthetic agents take effect and before the patient is successfully intubated and mechanically ventilated. High flow respiratory support refers to the delivery of heated and humidified respiratory gases to a patient via a non-sealing patient interface (e.g. nasal cannula) at high flow rates that when the patient is spontaneously breathing, are generally intended to meet or exceed inspiratory demand of a patient.

Once pre-oxygenated, anaesthetic agents are delivered to a patient to sedate the patient prior to intubation. Post intubation, anaesthetic agents are also delivered to maintain the anaesthetized state of the patient during a medical procedure. This delivery of anaesthetic agents can be done intravenously or via inhalation of aerosols/vapor—the latter may be achieved by the use of an anaesthesia machine. A system configured for anaesthetic procedure typically includes an anaesthesia machine which includes a rebreathing system in which expired gases from the patient are returned to the machine. The anaesthesia machine provides the anaesthetic agents to sedate the patient and/or keep the patient sedated via a sealing mask placed on the patient. Once sedated, patients are intubated and then mechanically ventilated by the anaesthesia machine (anaesthetic ventilation) that assists or replaces spontaneous breathing.

Provision of various modes of respiratory support rarely occurs in a straightforward sequential fashion. In the clinical setting it is often necessary to switch between modes of support e.g. to ensure adequate oxygenation during anaesthetic induction and intubation. Since each different mode of respiratory support may require use of different patient interfaces, e.g. non-sealing, sealing, face mask, endotracheal tube etc., this can require multiple inspiratory gas flow conduits providing flows of gases from multiple gas sources or respiratory support systems. It may be desirable to simplify the clinical setting by reducing the number of tubes and/or connections (and/or connection/disconnection actions) while still enabling the clinician to switch between different modes of respiratory support as may be appropriate to achieve desired clinical outcomes for the patient.

A reference herein to a patent document or any other matter identified as prior art, is not to be taken as an admission that the document or other matter was known or that the information it contains was part of the common general knowledge.

### SUMMARY OF INVENTION

Viewed from one aspect, the present disclosure provides a device for providing respiratory support to a patient, the device comprising: (a) a first gas flow path; (b) a second gas flow path; (c) a first gases port configured to receive gases from either of the first gas flow path and the second gas flow path; and (d) a switching mechanism operable to switch flow to the first gases port between the first gas flow path and the second gas flow path; wherein gases from the first gases port are provided to the patient for respiratory support.

In some embodiments, operation of the switching mechanism to allow flow from one of the gas flow paths to the first gases port prevents flow from the other of the gas flow paths to the first gases port.

In some embodiments, the first gases port is couplable with a first conduit providing gases to the patient through a second patient interface.

In some embodiments, the device comprises a second gases port which is couplable with a second conduit configured to receive expired gases from the patient. The device may comprise a first patient interface such as e.g. a face mask configured to receive expired gases from the patient for return to the device through the second conduit.

In some embodiments, application of the first patient interface to the patient causes the switching mechanism to allow flow to the first gases port from the first gas flow path. In some embodiments, application of the first patient interface to the patient may be determined by detecting an increase in one or more parameters of gases an expiratory gas flow path between the second patient interface and the device, the parameters selected from a group including, gas pressure, CO2, O2, flow rate, temperature and humidity. In some embodiments, the first patient interface is a face mask comprising a sealing cuff having a sensor for determining pressure within the cuff, and wherein application of the face mask to the patient is determined by an increase in cuff pressure.

In some embodiments, the first patient interface comprises one or more sensors configured to determine when the second patient interface has been applied to the patient. In some embodiments, the one or more sensors of the first patient interface include one or more of an optical sensor, a proximity sensor and a thermal sensor.

In some embodiments, the device comprises an endotracheal tube (ETT) or laryngeal mask airway (LMA) couplable with the first and second conduits via a wye-piece connector and functions as both the first patient interface and the second patient interface. In some such embodiments, the device may be configurable for use as a ventilator.

In some embodiments, removal of the first patient interface from the patient causes the switching mechanism to allow flow to the first gases port from the second gas flow path.

In some embodiments, when gases flow to the first gases port from the second gas flow path, the device is operable to provide gases to the patient through a second patient interface being a non-sealing patient interface such as e.g. a nasal cannula.

In some embodiments, the switching mechanism comprises a switching element which, when the switching mechanism operates to allow flow from one of the gas flow paths to the first gases port, is configured to prevent flow from a flow source to the other gas flow path.

In some embodiments, the switching mechanism comprises one or more valves upstream of a junction between the first gases flow path and the second gases flow path which, when the switching mechanism operates to allow flow from one of the gas flow paths to the first gases port, are configured to prevent backflow in the other gas flow path.

In some embodiments, the switching mechanism may be operatively coupled with one or more flow meters configured to stop a gases flow to the gas flow path that does not provide a flow of gas to the first gases port according to operation of the switching mechanism.

In some embodiments, the switching mechanism is operatively coupled with one or more flow meters operable to control a gases flow to the gas flow path providing a flow of gases to the first gases port according to operation of the switching mechanism.

In some embodiments, the switching mechanism is operatively coupled with one or more flow meters operable to control a gases flow to the gas flow path providing a flow of gases to the first gases port according to operation of the switching mechanism, wherein the one or more fixed flow meters control the gases flow at one or more pre-set gas flow rates.

In some embodiments, the device comprises a controller in operable communication with the switching mechanism and operable to the device to deliver respiratory support to the patient. In some embodiments, the controller may receive inputs from the one or more sensors for determining if the first patient interface has been applied to the patient.

In some embodiments, upon determination that the first patient interface has been applied to the patient, the controller automatically controls the device to provide respiratory support in a first mode; and upon determination that the first patient interface has been removed from the patient, the controller automatically controls the device to provide respiratory support in a second mode.

In some embodiments, the controller may receive inputs from one or more sensors in one or more locations selected from a group including: in an inspiratory flow path providing gases to a patient; in the device between the first gases port and a junction where returned gases in the device meet with fresh gas supply; in an expiratory flow path returning gases from the patient to the device; in the device between a second gases port receiving returned gases from the patient and the junction; and in a gases flow downstream of a flow generator or flow mixer of the device. The one or more sensors may be a sensor type selected from a group including but not limited to pressure sensors, flow rate sensors and O2 sensors.

In some embodiments, the controller may receive inputs from one or more CO2 sensors in one or more locations selected from a group including: an expiratory flow path returning gases from the patient to the device; and in a device having a rebreathing circuit and CO2 absorber, in the device between a second gases port receiving returned gases from the patient and the CO2 absorber. In some embodiments, the controller determines the one or more locations containing a CO2 concentration higher than ambient air is associated with the patient interface that is applied to the patient.

In some embodiments, the controller may receive inputs from multiple sensors to determine if the first patient interface has been applied to the patient to mitigate erroneous switching between the first mode and the second mode.

In some embodiments, the controller may control the device to provide respiratory support with a signature flow element, and wherein the controller looks for the signature flow element in returned gases to determine that the first patient interface has been applied to the patient. The signature flow element may comprise, for example, an oscillation in one or more of the frequency, amplitude and profile of one or more of pressure, flow rate and O2 concentration of the gases provided to the patient.

In some embodiments, the controller may receive sensor signals from one or more sensors comprising one or more pressure and/or flow rate and/or gas concentration sensors in an inspiratory flow path and/or an expiratory flow path to control switching from the second mode to the first mode, and optionally wherein the same or different one or more sensor signals may be received by the controller to control switching from the first mode to the second mode. The controller may be configurable to require more sensor conditions to be met before switching from the first mode to the second mode, or vice versa. This may be configurable according to user preference.

In some embodiments, wherein the controller may control the device to provide a residual flow of gas in one or both of the first gas flow path and the second gas flow path for continuous or regular monitoring of gas. In some embodiments the residual flow may be from e.g. about 0.5 L/min to about 5 L/min.

In some embodiments, the device may comprise one or more sensors sensing one or more characteristics of gas selected from a group including: pressure, flow rate and gas species concentration.

In some embodiments, wherein the controller may apply a timing control, wherein upon detection of a condition that should trigger a mode switching, the controller applies a delay before controlling the switching elements to cause the mode switch. In some embodiments the controller may control a user interface to provide one or more of an audible and visible countdown indicator of when the controller will switch control of the device between the first mode and the second mode. In some embodiments, the controller may be configured to abandon an automated switch between modes upon receipt of a user input to cancel during the delay.

In some embodiments, wherein the controller may control a user interface providing one or both of an audible and visible mode indication representing a current operating mode of the device. In some embodiments, wherein the controller may the user interface may comprise one or more audible and/or visible output elements located on, at or near a gas conduit or patient interface and providing the mode indication.

In some embodiments, the device may comprise a user interface in operable communication with the controller and configured to receive input from a user corresponding to one or more parameters of the respiratory support to be provided to the patient. The parameters may comprise one or more parameters for a rebreathing mode of respiratory support and/or a high flow mode of respiratory support. Alternatively or additionally, the parameters may comprise one or more of flow rate, composition, pressure, temperature and humidity of gases to be provided to the patient.

In some embodiments, the controller may be configured to receive a user input causing the switching mechanism to switch flow to the first gases port between the first gas flow path and the second gas flow path. The user input may be received from one or more of: (a) a touch screen display; (b) a wired or wirelessly coupled remote unit; (c) a foot operated pedal or switch; and (d) a physical switch provided on the device.

In some embodiments, the first gas flow path comprises a rebreathing gas flow path which receives and processes expired gases returned from the patient. Processing expired gases returned from the patient may comprise recirculating the expired gases in the first gas flow path. In some embodiments, the device may be operable to provide one or both of anaesthesia and ventilatory respiratory support via the rebreathing gas flow path.

In some embodiments, the second gas flow path comprises a high flow gas flow path. In some embodiments, the device comprises a flow source configured to generate the flow of gases in the second gas flow path.

In some embodiments, the first gases port is a common gases outlet port which is couplable with a first conduit for delivering gases to a patient from either the first gas flow path or the second gas flow path.

In some embodiments, a housing in which the first gas flow path and the second gas flow path are provided, the housing providing a first gases coupling defining the first gases port with which a first conduit is couplable.

In some embodiments, the device comprises a humidifier configured to condition gases to a pre-determined temperature and/or humidity before delivery to the patient via one or both of the first and second gas flow paths.

In some embodiments, the device may be operable to provide a flow of gases in the second gas flow path at a flow rate that is selectable from an available range of about 20 L/min to about 100 L/min. Alternatively, the device may be operable to provide a flow of gases in the second flow path at a flow rate that is selectable from a plurality of available fixed flow rates including at least 0 L/min, 40 L/min and 70 L/min.

In some embodiments having a second gases port configured to receive expired gases from the patient, the device may comprise a CO2 absorber configured to treat expired gases returned from the patient in the first gas flow path. This may be provided before recirculating gases to the patient in the first gas flow path.

In some embodiments, the device comprise one or more of the following features in the first gas flow path: (a) a pressure limiting valve configured to maintain substantially stable pressure in the first gas flow path; (b) a variable volume for displacement of gases in the first gas flow path; (c) a replenishing gas flow for replenishing anaesthetic gas delivered to the patient in the first gas flow path; (d) a gas mixer; (e) a vaporizer for vaporizing volatile anaesthetic agents into gas in the first gas flow path; and (f) a flow rate limiter configured to limit flow in the first gas flow path to about 15 L/min.

In some embodiments, the device may comprise one or more gases supply ports configured to receive a supply of one or both of: (a) a breathing gas for delivery to the patient by the first or the second gas flow path; and (b) an anaesthetic gas for delivery to the patient by the first gas flow path.

In some embodiments, the device may be configured for coupling to a power source comprising a general power outlet or a battery.

In some embodiments having a second gases port configured to receive expired gases from the patient, the device may be configured for operation: (a) in a rebreathing mode wherein gases flow from the first gas flow path to the first gases port and are provided to the patient, and expired patient gases are returned to the device via the second gases port; and (b) a high flow mode wherein gases flow from the second gas flow path to the first gases port and are provided to the patient without expired patient gases being returned to the device. In some embodiments, the rebreathing mode may comprise an anaesthetic rebreathing mode for provision of anaesthesia to the patient. Alternatively or additionally, the device may be configurable for use in a ventilation rebreathing mode, wherein gases flow from the first gas flow path to the first gases port and are provided to the patient through an ETT or LMA from which expired patient gases are returned to the device via the second gases port. The device may be operable to provide for automatic rebreathing e.g. by use of bellows in the device, or for manual rebreathing by use of a manually operated bag ventilator.

Viewed from another aspect, the present disclosure provides a device for providing respiratory support to a patient, the device comprising: a first gas flow path; a second gas flow path; a first gases port configured to receive gases from either of the first gas flow path and the second gas flow path; and a switching mechanism operable to switch flow to the first gases port between the first gas flow path and the second gas flow path; wherein gases from the first gases port are provided to the patient for respiratory support; and wherein the device comprises a controller configured to receive inputs from one or more sensors for determining if a condition exists to trigger switching the flow to the first gases port between the first gas flow path and the second gas flow path, wherein the one or more sensors are provided in one or more locations selected from a group including: in an inspiratory flow path providing gases to a patient; in the device between the first gases port and a junction where returned gases in the device meet with fresh gas supply; in an expiratory flow path returning gases from the patient to the device; in the device between a second gases port receiving returned gases from the patient and the junction; and in a gases flow downstream of a flow generator or flow mixer of the device.

In some embodiments, the device comprises a second gases port configured to receive gases returned from the patient, and the condition comprises the controller determining if a sealing patient interface in fluid communication with second gases port is, or is not, applied to the patient.

Viewed from another aspect, the present disclosure provides a device for providing respiratory support to a patient, the device comprising: a first gas flow path; a second gas flow path; a first gases port configured to receive gases from either of the first gas flow path and the second gas flow path; and a switching mechanism operable to switch flow to the first gases port between the first gas flow path and the second gas flow path; wherein gases from the first gases port are provided to the patient for respiratory support; and wherein the device comprises a controller configured to control the device to provide respiratory support with a signature flow element, and wherein the controller looks for the signature flow element in gases returned from the patient to the device to determine if a patient interface has been applied to the patient.

In some embodiments, the signature flow element may comprise an oscillation in one or more of frequency, amplitude and profile of one or more of pressure, flow rate and O2 concentration of the gases provided to the patient.

It is to be understood each of the various aspects described herein may incorporate one or more features, modifications and alternatives described in the context of one or more other aspects and may include one or more features, modifications and alternatives of any of the embodiments described below, as appropriate. For efficiency, such features, modifications and alternatives have not been repetitiously disclosed for each and every aspect although one of skill in the art will appreciate that such combinations of features, modifications and alternatives disclosed for some aspects and embodiments apply similarly for other aspects and are within the scope of and form part of the subject matter of this disclosure.

### BRIEF DESCRIPTION OF DRAWINGS

The invention will now be described in greater detail with reference to the accompanying drawings in which like features are represented by like numerals. It is to be understood that the embodiments shown are examples only and are not to be taken as limiting the scope of the invention as defined in the provisional claims appended hereto.

FIG. 1 is a schematic diagram showing components of a prior art anaesthesia machine.

FIG. 2 is a schematic diagram of a prior art ventilator.

FIG. 3 is a schematic diagram of components of a prior art high flow system.

FIG. 4 is a schematic illustration showing components in a device for providing respiratory support to a patient according to an embodiment of the disclosure.

FIG. 5 is a schematic illustration showing a modification of the device in FIG. 4 in which there are switching elements.

FIG. 6 is a schematic illustration of a device for providing respiratory support having switching mechanism comprising a switching element located between a gas source and the first and second flow paths according to an embodiment of the disclosure.

FIG. 7 is a modified example of the device in FIG. 6 in which the humidifier is a separate component provided outside the device.

FIG. 8 is another modified example of the device in FIG. 6 in which the humidifier is a separate component provided outside the device.

FIG. 9 is a schematic illustration of a device for providing respiratory support having a switching mechanism comprising a flow rate selector for the second gas flow path according to another embodiment of the disclosure.

FIG. 10 is a schematic illustration of a device for providing respiratory support having a switching mechanism comprising a set of flow rate selectors common to both the first gas flow path and the second gas flow path according to another embodiment of the disclosure.

FIG. 11 is a schematic illustration of a device for providing respiratory support having a switching mechanism comprising separate sets of flow rate selectors for each gas flow path according to another embodiment of the disclosure.

FIG. 12 is a schematic illustration of a device for providing respiratory support showing possible locations for sensors sensing characteristics of gases in the inspiratory flow path and the expiratory flow path.

FIG. 13 is a schematic illustration of a device providing respiratory support showing possible locations for sensors sensing characteristics of gases when a residual flow is provided.

FIGS. 14 to 16 illustrate use of multiple patient interfaces in the delivery of different modes of respiratory support according to embodiments of the present disclosure.

FIG. 17 is a schematic illustration of a connector that may be used to facilitate interchanging of components for delivery of different modes of respiratory support according to embodiments of the present disclosure.

FIG. 18 is a schematic illustration of another connector for use according to embodiments of the present disclosure.

FIG. 19 is a schematic illustration of another connector for use with a nasal cannula used to deliver different modes of respiratory support according to embodiments of the present disclosure.

FIG. 20 is a schematic illustration of a connector which is a variation on the connector of FIG. 19.

FIG. 21 is a schematic illustration of a connector which is another variation on the connector of FIG. 19.

### DETAILED DESCRIPTION

Embodiments of the invention are discussed herein by reference to the drawings which are not to scale and are intended merely to assist with explanation of the invention.

### Components of Anaesthesia Machines

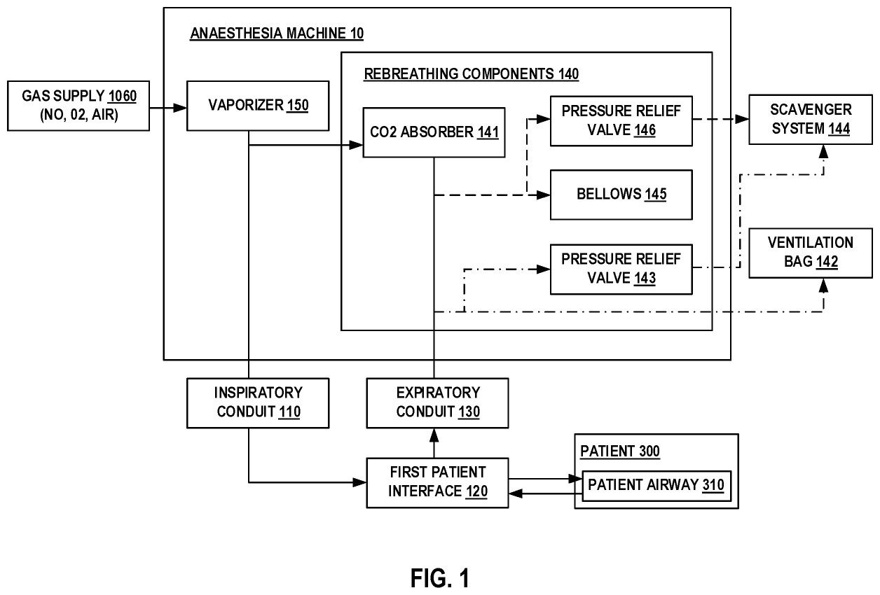

FIG. 1 is a schematic diagram showing components of an anaesthesia machine 10, which is configurable to receive a gas supply 1060 for delivering a respiratory support to a patient 300 through piped connections known in the art. The gas supply 1060 may include one or more of an anaesthetic gas (e.g. nitrous oxide (NO)), oxygen (O₂) and air supply. The air supply may be ambient air. Flow meters may be incorporated into the gas supply 1060, or placed upstream of the anaesthesia machine 10, or incorporated into it, to control the flow of gases through the machine. Flow meters may be manually controlled and/or precision controlled by a controller of the anaesthesia machine.

A breathing circuit delivers gases to the patient 300 and returns expired gases to rebreathing components 140. The breathing circuit may comprise corrugated tubing, valves and one or more patient interfaces for directing gases into the patient's airway and removing expired gases. In the schematic illustration of FIG. 1 the breathing circuit is simplified and denoted as including (but not limited to) inspiratory conduit 110 and first patient interface 120 which directs gas into the airway 310 of patient 300 and expiratory conduit 130 which collects expired gases. Thus, first patient interface 120 may be a sealing interface such as a sealing mask or endotracheal tube and may be configured to direct expired gases from the patient 300 to an expiratory flow path 130 which returns the expired gases to rebreathing components 140 of the anaesthesia machine 10. The inspiratory and expiratory conduits may be connected to the patient interface by a wye piece connector.

One or more vaporizers 150 convert volatile anaesthetics such as isoflurane and sevoflurane from liquid to vapour, and control introduction of these agents into the breathing circuit in accurately controlled concentration and dosages as required by the user, such as an anaesthetist clinician. Vaporizers 150 may be manually controlled and/or precision controlled by a controller of the respiratory apparatus. In some embodiments, vaporizers 150 feed into the rebreathing components 140.

Integrated into the anaesthesia machine 10 is a ventilation system which ventilates patient 300 during induction and after administration of anaesthetic agents to achieve ongoing anaesthesia. A manual ventilation bag 142 may be used by a clinician e.g. during induction (dot-dash lines inside rebreathing components 140) when volatiles are being delivered and prior to the patient being intubated. The compliance of the ventilation bag 142 enables the patient to breathe in and out a fixed volume of gas through a sealing first patient interface 120 in the form of a face mask. Once intubated, the ventilation mode changes from manual to mechanical, effectively isolating the manual ventilation bag 142 and associated pressure relief valve 143 from the rebreathing components 140 so that ventilation occurs via a mechanical system (dash lines inside rebreathing components 140). This may involve a collapsible bellows 145 and/or electronically actuated valves (under control of a controller of the anaesthesia machine 10) that control the tidal volume and timing of breaths delivered to the patient through a sealing first patient interface 120 in the form of an endotracheal tube or a sealing face mask. Gases provided to the patient may be pressure and/or volume controlled, and/or flow controlled by the mechanical system. Pressure relief valves 143, 146 provide for release of excess gases from the rebreathing components 140 (arising from fresh gas flow from vaporizers 150 and returned expired patient gases) while preventing ambient air from entering the breathing circuit. Manual ventilation via ventilation bag 142 may also be used after intubation, if desired.

Rebreathing components 140 provide a gas recirculation system in which exhaled gases from the patient are treated as they flow around the circuit and are then re-inhaled. This provides advantages by reusing oxygen and volatiles that are present in the expired gas flow from the patient, reducing costs as well as the presence of anaesthetic agents in the atmosphere. Expired gases in the rebreathing components 140 are passed through CO₂ absorber 141 which may include a canister containing soda lime (or another CO₂ absorbing substance). The soda lime (a mixture of NaOH & Ca(OH)2) acts as a CO₂ scrubber to remove CO₂ before gases in the rebreathing components 140 re-enter the inspiratory conduit 110. Additionally, gases from pressure relief valves 143, 146 are directed via an exhaust (not shown) to an external scavenger system 144 which filters and collects anaesthetic gases from the gas flow.

It is to be understood that further features may be provided as part of an anaesthesia machine 10, such as e.g. patient monitoring, suction, pressure gauges, regulators and “pop-off” valves to protect the patient and components of the machine from high pressure gases, as are known in the art. For simplicity, these are not included in the example shown.

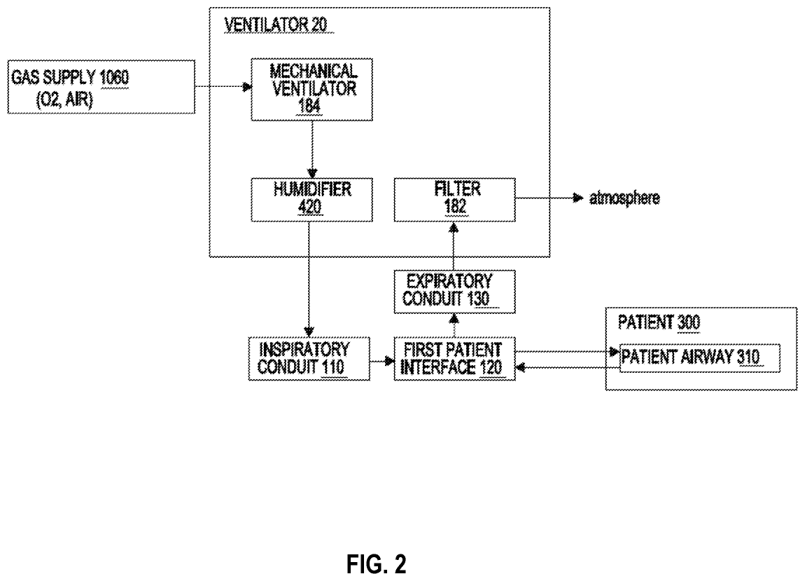

FIG. 2 is a schematic diagram of a ventilator 20 which may be used in an Intensive Care Unit (ICU). Ventilator 20 ventilates a patient with gases from gas supply 1060 and often with active humidification by humidifier 420 which is configured to heat and humidify gases delivered to the patient's airway 310. While humidifier 420 is shown as part of ventilator 20 in FIG. 2, this need not be the case. Gases from ventilator 20 may be humidified by a humidifier provided separately from the ventilator, humidifying gases from the ventilator before they reach firsts second patient interface 120. A ventilator 20 can support the patient's own breathing or replace it by delivering respiratory gases to a patient that are controlled to replicate ‘normal’ inhalation and exhalation breathing phases. Mechanical ventilator 184 may include a flow modulator and/or blower, and controls the pressure, volume and breathing rate of breathing gases delivered through inspiratory conduit 110 which is delivered to the patient by a sealing first patient interface 120. The sealing first patient interface may be invasive (e.g. endotracheal tube or laryngeal mask airway (LMA)) or non-invasive (e.g. sealing face mask). Exhaled gases leave the patient via the first patient interface and expiratory conduit 130 where they are treated e.g. by filter 182 and released to atmosphere. In some non-invasive ventilation systems expired gases exit through a vent or exhaust ports in the patient interface 120 or the expiratory conduit which enables expired gases to exit to atmosphere without returning to the ventilator apparatus 20.

### Components of High Flow Systems

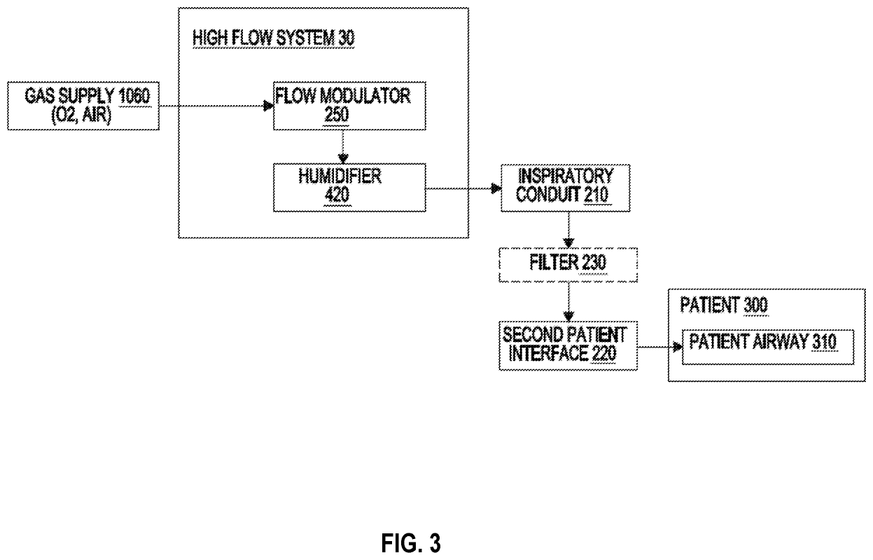

FIG. 3 is a schematic diagram of components of a high flow system 30 which is configurable to receive gas from a gas supply 1060 for delivering high flow respiratory support to a patient 300. The gas supply 1060 may be one or more of an anaesthetic gas (e.g. nitrous oxide (NO)), oxygen (O2) or air supply, preferably an O2 and/or air supply. The air supply may be ambient air. High flow system 30 has a flow modulator 250 configured to generate gas flows that are passed through a humidifier 420 which is configured to heat and humidify gas flows generated by the flow modulator 250. In some embodiments, the flow modulator 250 may comprise a gas supply 1060 as described below or a flow source such as a fan or blower. The humidified high gas flow is delivered to the patient 300 by a second inspiratory conduit 210 and a non-sealing second patient interface 220. This may comprise a nasal cannula, which directs the high flow of breathing gases into the patient's airway 310 through one or both nares. An optional filter 230 may be provided between the inspiratory conduit 210 and the second patient interface 220 so that components of the breathing circuit upstream from the filter can be reused without risk of contamination by any inadvertently captured expired gases by the second patient interface 220. Relevantly, filter 230 may be provided elsewhere in the flow path such as e.g. between humidifier 420 and inspiratory conduit 210. In some examples, a filter may be associated with a cannula of a non-sealing second patient interface 220 and/or in expiratory flow path 130.

In some configurations, the flow modulator 250 is configured to provide gases to the patient through high flow system 30. In some embodiments, the flow modulator comprises a gas generation means, for example a blower adapted to receive gases from the environment outside of the high flow system 30 and propel them through the high flow system 30. In some configurations, the flow modulator 250 may comprise a source available from a hospital gas outlet or wall supply (e.g. oxygen or air), or one or more containers of compressed air and/or another gas and one or more valve arrangements adapted to control the rate at which gases leave the one or more containers. In some configurations, the flow modulator 250 may comprise an oxygen concentrator.

In this specification, “high flow” means, without limitation, any gas flow with a flow rate that is higher than usual/normal, such as higher than the normal inspiration flow rate of a healthy patient, or higher than some other threshold flow rate that is relevant to the context. It can be provided by a non-sealing respiratory system with substantial leak, for example happening at the entrance of the patient's airways. It can also be provided with humidification to improve patient comfort, compliance and safety. “High flow” can mean any gas flow with a flow rate higher than some other threshold flow rate that is relevant to the context—for example, where providing a gas flow to a patient at a flow rate to meet inspiratory demand, that flow rate might be deemed “high flow” as it is higher than a nominal flow rate that might have otherwise been provided. “High flow” is therefore context dependent, and what constitutes “high flow” depends on many factors such as the health state of the patient, type of procedure/therapy/support being provided, the nature of the patient (big, small, adult child) and the like. A person skilled in the art would appreciate, in a particular context what constitutes “high flow”. But, without limitation, some indicative values of high flow can be as follows.

In some configurations, high flow delivery of gases to a patient at a therapeutic flow rate may comprise a flow rate of greater than or equal to about 5 or 10 litres per minute (5 or 10 LPM or L/min). The therapeutic flow rate can be time-varying (e.g. oscillating). That is, the therapeutic flow can have a time-varying (e.g. oscillating) flow rate component. This time-varying flow rate can aid respiratory support by providing improved oxygenation and/or CO₂ clearance and/or reduce the risk of atelectasis which may in turn lead to more evenly distributed patient pressure throughout the lung.

In some configurations, high flow delivery of gases to a patient is at a flow rate of about 5 or 10 LPM to about 150 LPM, or about 10 LPM to about 120 LPM, or about 15 LPM to about 95 LPM, or about 20 LPM to about 90 LPM, or about 20 LPM to about 70 LPM, or about 25 LPM to about 85 LPM, or about 30 LPM to about 80 LPM, or about 35 LPM to about 75 LPM, or about 40 LPM to about 70 LPM, or about 45 LPM to about 65 LPM, or about 50 LPM to about 60 LPM. For example, according to those various embodiments and configurations described herein, a flow rate of gases supplied by embodiments of the systems disclosed, may comprise, but is not limited to, flows of at least about 5, 10, 15, 20, 30, 40, 50, 60, 70, 80, 90, 100, 110, 120, 130, 140, 150 LPM, or more, and useful ranges may be selected to be any of these values (for example, about 20 LPM to about 90 LPM, about 15 LPM to about 70 LPM, about 20 LPM to about 70 LPM, about 40 LPM to about 70 LPM, about 40 LPM to about 80 LPM, about 50 LPM to about 80 LPM, about 60 LPM to about 80 LPM, about 70 LPM to about 100 LPM, about 70 LPM to about 80 LPM). Thus, ‘high flow’ or ‘high flow respiratory support may refer to the delivery of gases to a patient at a flow rate of between about 5 or 10 LPM and about 100 LPM, or between about 15 LPM and about 95 LPM, or between about 20 LPM and about 90 LPM, or between about 25 LPM and about 85 LPM, or between about 30 LPM and about 80 LPM, or between about 35 LPM and about 75 LPM, or between about 40 LPM and about 70 LPM, or between about 45 LPM and about 65 LPM, or between about 50 LPM and about 60 LPM.

In “high flow” the gas delivered will be chosen depending on for example the intended use of a therapy or support. Gases delivered may comprise a percentage of oxygen. In some configurations, the percentage of oxygen in the gases delivered may be about 15% to about 100%, 20% to about 100%, or about 30% to about 100%, or about 40% to about 100%, or about 50% to about 100%, or about 60% to about 100%, or about 70% to about 100%, or about 80% to about 100%, or about 90% to about 100%, or about 100%, or 100%.

Flow rates for “High flow” for premature/infants/paediatrics (with body mass in the range of about 1 to about 30 kg) can be different. The flow rate can be set to about 0.4 LPM/kg to about 8 LPM/kg with a minimum of about 0.5 LPM and a maximum of about 70 LPM. For patients under 2 kg maximum flow may be set to 8 LPM. Oscillating flow may be set to 0.05-2 L/min/kg with a preferred range of 0.1-1 L/min/kg and another preferred range of 0.2-0.8 L/min/kg.

High flow may be used as a means to promote gas exchange and/or respiratory support through the delivery of oxygen and/or other gases, and through the removal of CO₂ from the patient's airways. High flow may be particularly useful prior to, during or after a medical procedure. Further advantages of high gas flow can include that the high gas flow increases pressure in the airways of the patient, thereby providing patency support that opens airways, the trachea, lungs/alveolar and bronchioles. The opening of these structures enhances oxygenation, and to some extent assists in removal of CO₂.

The increased pressure can also keep structures such as the larynx from blocking the view of the vocal chords during intubation. When humidified, the high gas flow can also prevent airways from drying out, mitigating mucociliary damage, and reducing risk of laryngospasms and risks associated with airway drying such as nose bleeding, aspiration (as a result of nose bleeding), and airway obstruction, swelling and bleeding.

In this specification, the terms subject and patient are used interchangeably. A subject or patient may refer to a human or an animal subject or patient.

In this specification, it is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

### Overview

Embodiments of the present disclosure relate to a device for providing respiratory support to a patient, which provides for switching between different forms or modes of respiratory support provided by a single apparatus. Such a device may be desirable and/or useful for a clinician user who may desire to switch between different forms of respiratory support delivered to a patient.

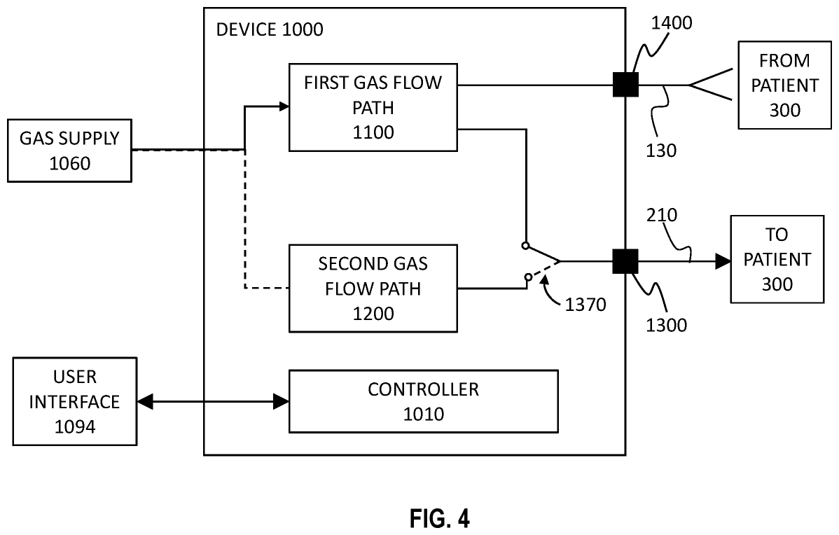

FIG. 4 is a schematic illustration showing components of a device 1000 for providing respiratory support to a patient. The device comprises first gas flow path 1100, a second gas flow path 1200 and a first gases port 1300 configured to receive gases from either of the first gas flow path and the second gas flow path. The device further comprises a switching mechanism 1370 operable to switch flow to the first gases port 1300 between the first gas flow path 1100 and the second gas flow path 1200. In uses where the first gases port 1300 is the port through which inspiratory gases are provided to the patient, the first gases port may be regarded as a common gases port for delivery of inspiratory gases to the patient.

In some embodiments, the device 1000 comprises a controller 1010, and operation of the switching mechanism 1370 may be triggered by the controller detecting of one or more conditions of operation of the device and/or by the controller receiving a user input, as will be described below. Thus, in some embodiments the device 1000 comprises a user interface 1094 which may comprise e.g. a touchscreen and/or display device and/or monitor with keyboard and/or other input actuators (buttons, knobs, dials etc) in operative communication with controller 1010.

Operation of the switching mechanism 1370 to allow flow from one of the gas flow paths to the first gases port 1300 may be referred to herein as to “enable” that flow path in that the switch enables flow to occur between the enabled flow path and the first gases port 1300. In some embodiments operation of the switching mechanism 1370 to allow flow from one of the gas flow paths to the first gases port 1300 also prevents flow from the other of the gas flow paths to the first gases port. The gas flow path from which flow to the first gases port 1300 is prevented may be referred to herein as “disabled” in that the switch does not enable or allow flow to occur between the disabled flow path and the first gases port 1300.

FIG. 4 also shows a gas supply 1060 for supplying gases to the first gas flow path 1100 and/or second gas flow path 1200, although it is to be understood that more than one gas supply may be provided in many embodiments. The gas supply 1060 shown in FIG. 4 is exemplary only and provided solely for context to show that at least one gas is provided to the device for the provision of respiratory support to the patient 300.

In some embodiments, the first gases port 1300 may be regarded as a gases outlet port with which a first conduit, such as an inspiratory conduit, may be coupled for provision of gas to the patient via a second patient interface as will be described below. FIG. 4 also shows a second gases port 1400 which is configured to be couplable with a second conduit, such as an expiratory conduit, configured to receive expired gases from the patient 300 via a first patient interface. The expired gases received at the second gases port 1400 are returned to the first gas flow path 1100 as will be described below.

However, in some uses there may be a change of flow direction through the first gases port 1300 when the switch occurs. For example, consider the second gases flow path 1200 is initially enabled with a flow of gas provided to the patient via a non-sealing second patient interface (e.g. cannula) for provision of high flow respiratory support. When the flow path to the first gases port 1300 is switched to enable the first gases flow path 1100, the non-sealing second patient interface could receive expired gases from the patient, with inspiratory flow provided from the second gases port 1400 and delivered through a sealing first patient interface (e.g. face mask). In this arrangement the flow directions outside the device 1000 are reversed with the non-sealing second patient interface and conduit 210 providing an expiratory flow path and the sealing first patient interface and conduit 130 providing an inspiratory flow path.

The switching mechanism 1370 may be provided by one or more components or combinations of components of the device which provide for switching between flow paths providing the flow of gas to the first gases port 1300. In the embodiment shown in FIG. 4, the switching mechanism 1370 comprises a switching element which has been operated to provide flow between the first gas flow path 1100 and the first gases port 1300. The broken lines show the alternative “switched” operation in which the switching element provides flow between the second flow path 1200 and the first gases port 1300. The switching mechanism 1370 may comprise a switching element such as a mechanical or pneumatic actuator, flow diverter, valve or the like and may be operated by a user of the device 1000 directly triggering the switching mechanism.

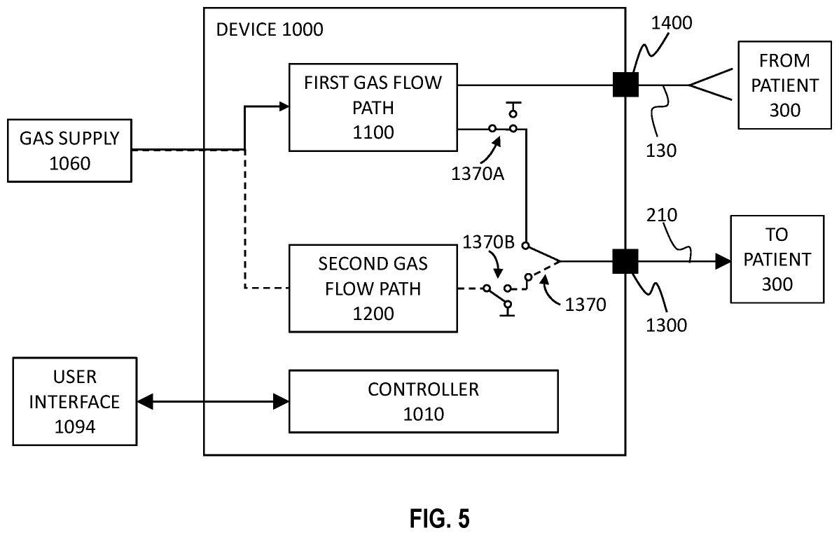

While the switching mechanism 1370 is shown as a single switch in FIG. 4, it is to be understood that the switching mechanism may comprise one or more switching elements 1370A and 1370B shown in the schematic illustration in FIG. 5. Here, a first switching element 1370A is provided for first flow path 1100 and a second switching element 1370B is provided for second flow path 1200. A switching element may comprise a shutoff or other valve operable under the control of a controller 1010 such as an electronic controller which may be a sensor driven automated controller. In some embodiments, the switching elements 1370A, B may include a user operable actuator as described herein in the context of an actuator for switching mechanism 1370.

The location of the switching mechanism 1370 in FIGS. 4 and 5 are representative only, and as will become apparent by reference to other examples provided herein, a switching mechanism may comprise one or more switching elements provided anywhere upstream of the junction between the first and second flow paths 1100, 1200 which supply the first gases port 1300, such as upstream of the components of the first and second gas flow paths 1100, 1200, and/or downstream of the elements of the first and second gas flow paths 1100, 1200.

The switching mechanism (and associated switching elements) may be operated directly by a user or indirectly, e.g. via a controller 1010 such as an electronic controller. Thus the switching mechanism 1370 may include a user operable actuator which enables a user to manually select the mode of operation and in turn, cause operation of the switching mechanism to enable or disable a gas flow path to the first gases port 1300, and/or a sensor driven automated controller that operates the switching mechanism 1370 (and other components of the device) as will be discussed below. Various switching elements may be operatively coupled to actuate substantially simultaneously, or in response to other switching elements or actuators or under control of the controller 1010 as will become apparent by reference to the non-limiting examples provided.

In some embodiments, the first gas flow path 1100 may be configurable to deliver breathing gas including one or more anaesthetic agents to the patient and the second gas flow path 1200 may be configurable to deliver breathing gas to the patient at a desired flow rate. Thus, first gas flow path 1100 may incorporate one or more components of an anaesthesia device 10 (FIG. 1) and/or a ventilation system 20 (FIG. 2) and the second gas flow path 1200 may incorporate one or more components of a high flow system 30 (FIG. 3). For simplicity, like numerals are utilised to denote such components throughout this disclosure.

In some embodiments, operation of the switching mechanism 1370 is undertaken to configure the device 1000 for different modes of operation for the provision of different forms of respiratory support to the patient. For example, when the switching mechanism 1370 is operated to enable the first gas flow path 1100, the device 1000 may be configured to operate in a first mode of operation which comprises a rebreathing mode in which expired gases from the patient are returned to the first gas flow path; and when the switching mechanism 1370 is operated to enable the second gas flow path 1200, the device may be configured to operate in a second mode of operation which may be a flow controlled mode without rebreathing. The second mode of operation may comprise a high flow mode. In some embodiments, when the switching mechanism 1370 is operated to enable the first gas flow path 1100, the device 1000 may be configured to operate in a first mode being an anaesthesia rebreathing mode or in a third mode being a ventilation rebreathing mode, as described previously in relation to FIG. 1. Thus, references to operation in the first mode are to be taken as also referring to the third mode which is also a rebreathing mode. Gases provided to the patient in this mode of operation may be pressure and/or volume controlled, and/or flow controlled. Ventilation may be triggered by patient effort or triggered by the ventilation device (e.g. anaesthesia machine) at a set respiratory rate.

While various embodiments are disclosed herein that prevent delivery of anaesthetic agents to the patient when the second flow path enabled, it is to be understood that yet other approaches may be deployed, as alternatives or in addition to the examples provided. For example, to avoid harm by release of anaesthetic agents while the second gas flow path is enabled, the device 1000 may inactivate release of anaesthetic agents to the patient by shutting down vaporizers or reducing their function to a level that is ineffective. Alternatively or additionally the device 1000 may inactivate delivery of anaesthetic agents. For example, the device may shut down vaporizers or reduce their function to a level that is ineffective, in the first gas flow path 1100. Alternatively or additionally the device may inactivate anaesthetic agents in a flow of gases delivered from the first gas flow path 1100 by use of a neutralizer in the first gas flow path that becomes activated when the device is operated with the gas flow path 1200 enabled, such that any anaesthetic agents that may be flowing through the device are rendered ineffective. While wasteful this may be an important safety measure.

When the first gas flow path 1100 is enabled, the device 1000 allows gas flow from the first gas flow path to the first gases port 1300 for operation of the device in the first mode. From here, a first conduit 210 provides gases from the first gases port 1300 to a second patient interface 220 (being an inspiratory patient interface) which directs the gases into an airway of the patient 300. In this arrangement, the second patient interface 220 providing gases to the patient may be a non-sealing interface such as a nasal cannula. A first patient interface being a sealing interface such as a face mask is configured to direct expired gases from the patient to second conduit 130 which returns the expired gases to the first gas flow path 1100 via second gases port 1400. Returned expired gases may be treated by rebreathing components 140 in the first gas flow path 1100 as described in relation to FIG. 1. When the first gas flow path 1100 is enabled, the device 1000 also prevents flow to the first gases port 1300 from the second gas flow path 1200, thereby rendering the second gas flow path “disabled”.

When the second gas flow path 1200 is enabled, the device 1000 allows gas flow from the second gas flow path to the first gases port 1300. Additionally, the device 1000 prevents flow of gas from the first gas flow path 1100 to the first gases port 1300. Thus, the first gas flow path 1100 is “disabled” so as to prevent delivery of anaesthetic gas which may include NO and vaporized anaesthetic agents to the patient 300. The first conduit 210 provides gases from the first gases port 1300 to a second patient interface 220 (being an inspiratory patient interface) which directs the gases into an airway of the patient 300. In this arrangement, the second patient interface 120 is a non-sealing interface such as a nasal cannula having one or more nasal prongs directing gases into one or both nares of the patient. There is no requirement for a first patient interface or second conduit to return expired gases to the device when the second gas flow path is enabled and the device is configured to operate in the second mode.

The device 1000 may be configured to receive a supply of gases including NO, O₂ and/or air from a gases supply 1060. One or more flow meters may be provided to control the flow rate of gases in the device 1000 to achieve desired respiratory support. One or more gas mixing elements may also be provided to combine the gases. The control of flow meters may be achieved by direct control performed by a user (e.g. actuating an actuator such as a rotary or linear switch directly altering flow through the flow meter), or by a user providing an input to a controller 1010 that is configured to provide precision control over e.g. electronically controlled flow meters within the device 1000.

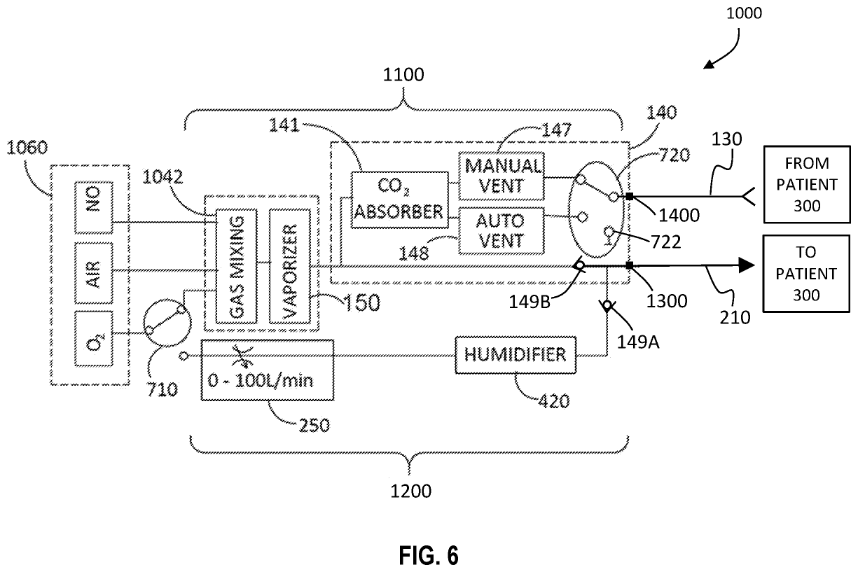

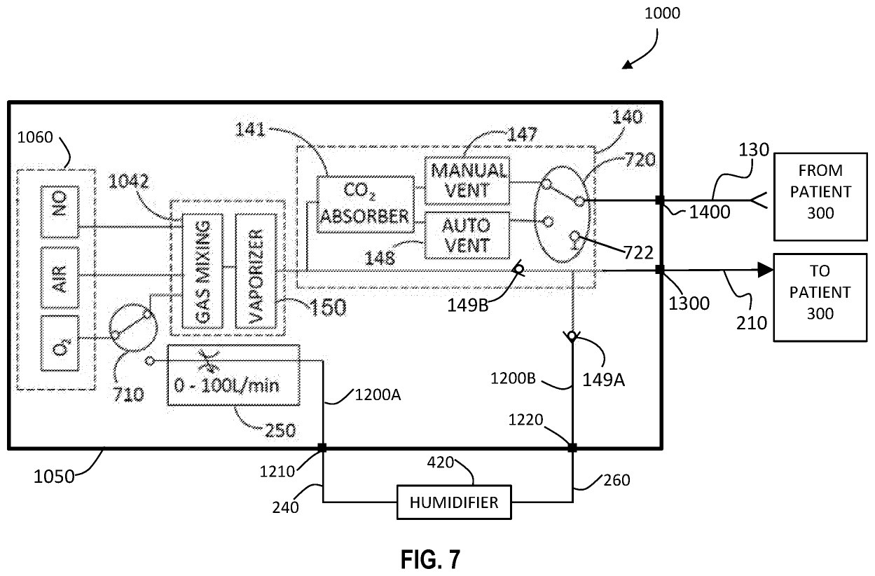

Flow meters may be incorporated into the gas supply 1060, or placed downstream of the gas supply to control the proportion of gases entering the first gas flow path 1100 and the second gas flow path 1200. Alternatively, the flow meters may be provided within the device 1000 (see FIGS. 6 and 7). Such flow meters can be manually controlled e.g. by a proportional valve with rotary actuator, or controlled by a controller 1010 of the device 1000. Safety features may be built in to limit flow rates and proportions of gases to be within safe limits, for example to ensure the flow rate of gases exiting the first gas flow path 1100 may not exceed 15 L/min in some embodiments and/or the FiO₂ does not decrease below 0.21.

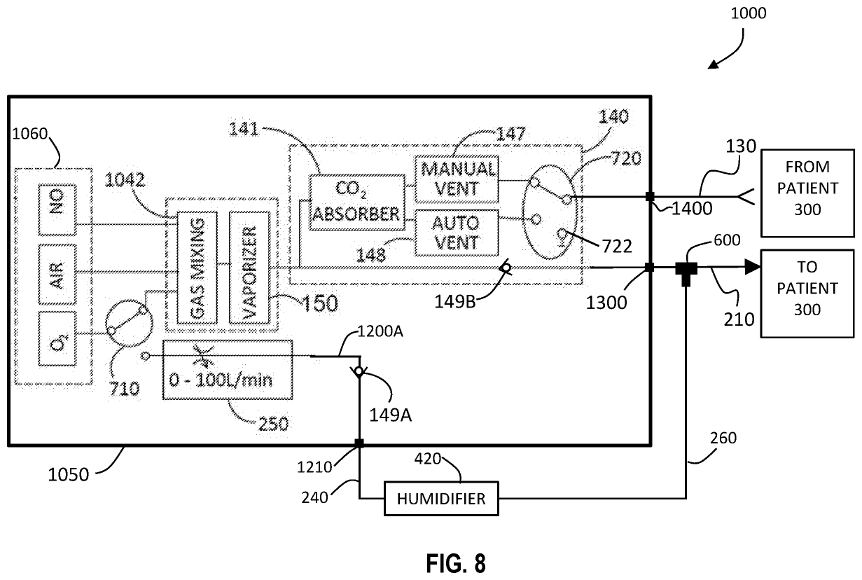

In some embodiments, the device 1000 may include a humidifier 420 (FIGS. 4A-7) configured to condition the gas to a pre-determined temperature and/or humidity before delivery to the patient. Preferably such conditioned gases are provided in the second gas flow path 1200 although it is contemplated that the humidifier 420 may be configured to condition gases to a pre-determined temperature and/or humidity before delivery to the patient via the first gas flow path 1100 when used in a ventilation rebreathing mode. Provision of the humidifier 420 within the device 1000 gives an advantage of streamlining set-up of the humidifier by integrating it into the daily routine of setting up the device 1000 for the provision of respiratory support to a patient. However, humidifier 420 need not be provided within device 1000. In some embodiments the device 1000 may be configured to operate with a humidifier 420 which is a separate component or apparatus as shown in FIGS. 7 and 8. Similar modifications may be made to the embodiments in FIGS. 5-7 for provision of a humidifier 420 outside the device however for conciseness these are not separately exemplified in the figures.

In FIGS. 7 and 8, device 1000 comprises a housing represented by solid line 1050 which contains elements of the first gas flow path 1100 and second gas flow path 1200 as shown in FIG. 6, but with humidifier 420 shown outside the device housing to represent a separate component or apparatus. A third gases port 1210 is provided downstream of the flow source 250 and is configured to enable fluid coupling between the first section 1200A of the second gas flow path and a third conduit 240 supplying gas to the humidifier 420.

In the arrangement of FIG. 7, humidified gases are returned to the device 1000 via fourth gases port 1220 which is configured to enable fluid coupling between a fourth conduit 260 containing humidified gases from humidifier 420, and the second section 1200B of the second gas flow path. In the arrangement of FIG. 8, the fourth conduit 260 does not couple with a gases port inside the device 1000. Instead, humidified gases in the fourth conduit 260 flow into the first conduit 210 via a junction or connector 600 such as a tee-piece connector. Connector 600 may form part of or be incorporated into first gases port 1300 or it may be provided downstream of the first gases port as shown in FIG. 8. It is to be noted that in the arrangement of FIG. 8, non-return valve 149A may be located upstream of the humidifier 420, inside the device housing 1050. Upstream placement of non-return valve 149A, relative to the humidifier 420 is also possible in FIG. 7 and in embodiments in which the humidifier is provided inside the device housing 1050. Non-return valves 149A, 149B may be provided to prevent backflow in the disabled flow path as described below.

The humidifier 420 may be controlled directly by a user providing manual inputs to a control interface provided with or on the humidifier when provided separately from the device 1000. Alternatively/additionally, the external humidifier may be in operative coupling with controller 1010 of the device such that operation of the device 1000 controls the performance of the humidifier 420 when provided as part of the device or when provide as a separate component that is external to the device. Thus, when the switching mechanism is operated to enable the second gas flow path, the controller 1010 may cause the humidifier 420 to increase the temperature and humidity of gases flowing in the second gas flow path 1200 to a pre-set target. The target temperature and humidity may be as programmed into the controller or selected by a user through the user interface 1094. In this sense controller 1010 may be regarded as a master controller which is operative to control all elements of the respiratory support provided to the patient.

When the switching mechanism is operated to switch flows to the first gases port 1300 from the second gas flow path 1200 to the first gas flow path 1100, the controller may cause humidifier 420 to enter a standby mode where the temperature is reduced below target temperature but the humidifier is not completely deactivated. This may improve performance when control is switched back to enabling the second gas flow path by enabling the humidified gases to reach target temperature and humidity faster. It is to be understood however that deactivation is also contemplated as a viable control during operation of the device when the first gas flow path is enabled (and the second gas flow path is disabled).

FIGS. 6 to 8 are schematic illustrations of a device 1000 in which the switching mechanism is located between a gas source 1060 and the first and second gas flow paths 1100, 1200. The switching mechanism comprises a first switching element 710 operable to direct flow of O₂ to the first gas flow path 1100 (which also receives a supply of Air and NO) for operation of the device in the first mode and to the second gas flow path 1200 for operation of the device in in the second mode. FIGS. 6 to 8 provide a schematic illustration depicting operation of such a switching mechanism to enable the first gas flow path 1100 for operation of the device 1000 in the first mode. Notably, in the first mode of operation a second switching element 720 is also operable, in a preferred embodiment, to permit the first gas flow path 1100 to perform in either a manual (147) or automatic/mechanical (148) ventilation/rebreathing mode. In the embodiment shown in FIGS. 6 to 8, the manual first mode has been selected. In some embodiments, the switching mechanism may also comprise one or more switching elements (not shown) operable to control flow of Air and/or NO to the first gas flow path 1100.

Second switching element 720 may be responsive to the first switching element 710. Therefore, when the first switching element 710 is switched to enable the second gas flow path 1200 for operation of the device in the second mode, O₂ is directed to the second gas flow path 1200 (represented by flow modulator 250 and humidifier 420) and the second switching element 720 can move to the lowest position 722 switching off both manual and automatic ventilation/rebreathing and preventing expired gases returned from the patient to second gases port 1400 from entering the rebreathing components 140 of first gas flow path 1100 and in turn, flowing to the first gases port 1300. When first switching element 710 is switched to enable the second gas flow path, O₂ is directed to the second gas flow path 1200. In the example of FIG. 8 gases from the second flow path 1200 enter the inspiratory conduit 210 downstream of first gases port 1300. Therefore operation of the first switching element 710 to enable the second gas flow path 1200 prevents flow of O₂ into the gas mixing element 1042 of first gas flow path 1100 while also preventing fresh gas flow to the patient 300 through first gases outlet 1300.

In addition to the switching mechanism which may comprise e.g. switching elements 710/730 and 720 in FIGS. 6 to 9, the device 1000 may comprise one or more non-return or other one way valves to prevent or control backflow in the system such as from the “disabled” gas flow path to the first gases port 1300. In the examples shown in FIGS. 6 to 9, when the switching mechanism is operated to switch flow to the first gases port 1400 from the first gas flow path 1100 to the second gas flow path 1200 non-return valve 149B prevents backflow from the first gases port into the first gas flow path. Conversely, when the switching mechanism is operated to switch flow to the first gases port 1300 from the second gas flow path 1200 to the first gas flow path 1100, non-return valve 149A prevents backflow from the first gases port 1400 to the second gas flow path 1200. Additional non-return valves may be implemented elsewhere in the flow paths of device 1000 as is commonly the case in gas flow systems and particularly in re-breathing circuits.

In some embodiments, including in the context of the examples already described, a desired flow rate of gas delivered to first gases port 1300 from the second gas flow path 1200 may be selectable from a range of from about 20 LPM to about 100 LPM by the user making a selection through a user interface 1094 of a controller 1010, or by manually operating a component of the switching mechanism or a flow controller of the device. However in some cases such as paediatric or neonatal patients, a lower range may be desired. In some embodiments, the desired flow rate may be selectable by the user from a plurality of available flow rates such as e.g. 0 LPM, 40 LPM and 70 LPM although it is to be understood that additional and/or different flow rates may be selectable within the high flow ranges disclosed herein.

Selection of the desired flow rate may be achieved by operation of a switching element which in some embodiments comprises a rate selector in the form of e.g. a knob, sliding switch, touch screen or other actuator which provides for user selection of the desired flow rate from a plurality or range of available flow rates.

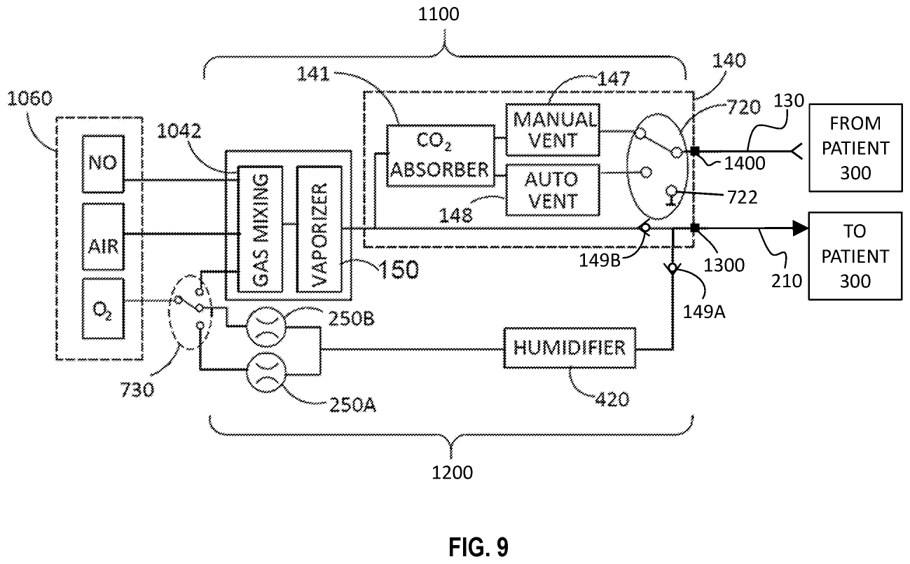

In some embodiments, the rate selector may comprise a rate switching element 730 as is schematically illustrated in FIG. 9. In some examples, rate switching element 730 may replace first switching element 710 however in some embodiments it may be desirable to provide rate switching element 730 in addition to first switching element 710 to avoid issues of delay in gas flow rate wind up when the second gas flow path 1200 is enabled. Preferably rate switching element 730 is operatively coupled with the second switching element 720 so that operation of the rate selector 730 to enable flow in the second gas flow path 1200 to the first gases port 1300 (including through humidifier 420 as shown) also prevents expired gases returned from the patient to second gases port 1400 from entering the rebreathing components 140 of first gas flow path 1100 and in turn, flowing to the first gases port 1300.

In the arrangement of FIG. 9, switching element 730 controls flow of O₂ to the first and second gas flow paths 1100, 1200. Thus, when the rate switching element 730 is operated to select one of the two predetermined flow rates associated with flow rate controller 250A (corresponding to e.g. 40 LPM) or flow controller 250B (corresponding to e.g. 70 LPM) there is flow of O₂ in the second gas flow path 1200 and the device 1000 is configured for operation in the second mode; when the rate switching element 730 is operated to select the top position shown in the figure, flow from the O₂ supply is directed to the first gas flow path 1100 and the device is configured for operation in the first mode. Rate switching element 730 and second switching element 720 may be operatively coupled such that operation of the rate selector 730 to enable the second gas flow path 1200 can activate switching of switching element 720 to the lowest position 722 preventing expired gases returned from the patient to second gases port 1400 from entering the rebreathing components 140 of first gas flow path 1100 and in turn, flowing to the first gases port 1300. Since operation of the rate switching element 730 to enable the second gas flow path 1200 prevents flow of O₂ into the gas mixing element 1042 of first gas flow path 1100, there is also no flow of fresh gas to the patient through first gases port 1300 from the first gas flow path 1100. In some embodiments, operation of switching element 730 may also shut off the NO/air supply to the second gas flow path 1200. This may be achieved e.g., by one or more operatively coupled shut off valves provided in the NO and/or air gas flow paths. In some embodiments, the switching mechanism may also comprise one or more switching elements (not shown) operable to control flow of Air and/or NO to the first and/or second gas flow paths 1100, 1200.

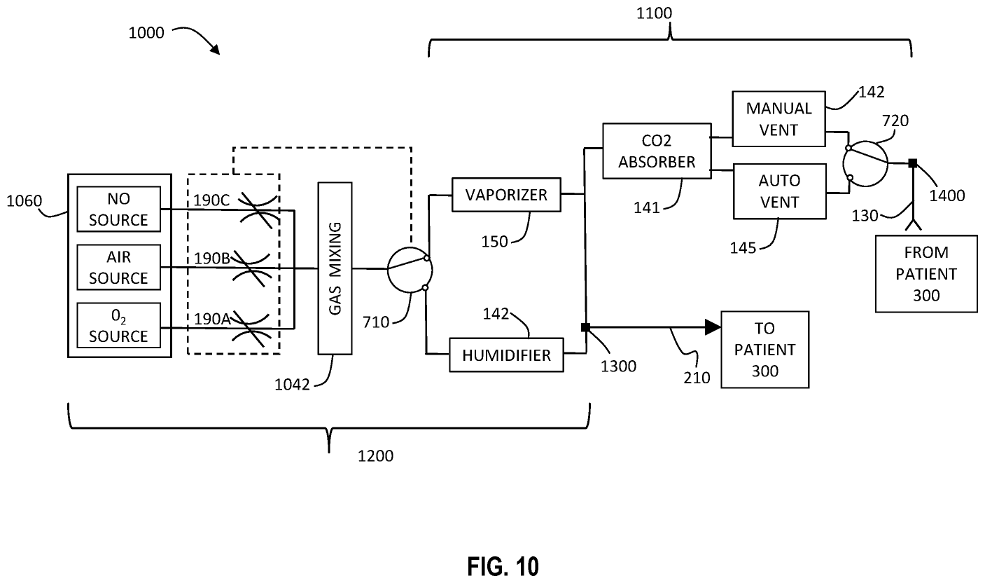

In some embodiments, the switching mechanism may comprise one or more flow rate selectors/flow meters 190A, 190B, 190C as shown in the schematic illustration of FIG. 10. In this example, a single set of flow meters 190A, 190B, 190C is provided to control flow of each of O2, Air and NO into a common gas mixing element 1042 from which both the first gas flow path 1100 and second gas flow path 1200 receive gases. In this arrangement, there is an operative coupling between the flow meters 190A, 190B, 190C and the switching mechanism comprising switching element 710 as shown by the broken line. As described previously, the switching element 710 is operable to switch the flow of gas to the first gases outlet 1300 between the first gas flow path 1100 and the second gas flow path 1200. In the arrangement of FIG. 10, when switching element 710 is operated to enable the first gas flow path 1100 (as in the illustrated configuration) the operative coupling between switching element 710 and flow meters 190A, 190B, 190C causes the flow meters to operate to deliver gases at flow rates suitable for achieving respiratory support in the first mode of operation. Conversely, when the switching element 710 is operated to enable the second gas flow path 1200, the operative coupling between switching element 710 and flow meters 190A, 190B, 190C causes the flow meters to operate to deliver gases at flow rates suitable for achieving respiratory support in the second mode of operation. The required flow rates for each flow meter 190A, 190B, 190C may be pre-set for operation in particular modes of operation. For example, when the first gas flow path 1100 is enabled, flow meters 190A, 190B, 190C may be configured to deliver a flow rate for the mixed gas that is suitable for provision of respiratory support in the first mode of operation (e.g. 15 L/min at the first gases outlet 1300). When the second gas flow path 1200 is enabled, flow meters 190A, 190B, 190C may be configured to deliver a flow rate for the mixed gas that is suitable for provision of respiratory support in the second mode of operation (e.g. 40 L/min or 70 L/min at the first gases outlet 1300). The flow rates provided by the flow meters 190A, 190B, 190C can be determined according to e.g. desired O2 and/or NO concentration. For example: to achieve 100% O2 concentration at a flow rate of 15 L/min flow meter 190A could be set at 15 L/min with zero flow through flow meters 190B, 190C. Alternatively or additionally, the NO source could be disabled when zero flow of NO is required, as an additional safety mechanism, e.g. when providing respiratory support in the second mode of operation. To achieve 50:50 ratio of O2: Air in the mixed gas at 15 L/min each of flow meters 190A and 190B could be set to provide 7.5 L/min. The flow from flow meter 190C could be adjusted according to the fraction of NO required in the mixed gas flow to the patient as would be decided by the clinician, with corresponding adjustments to flows from one or both of flow meters 190A,B according to clinical requirements. Alternatively or additionally, the flow meters 190A, 190B, 190C may be adjustable through manual operation or via a user interface 1094 in arrangements where the flow meters are in operative communication with a controller 1010.

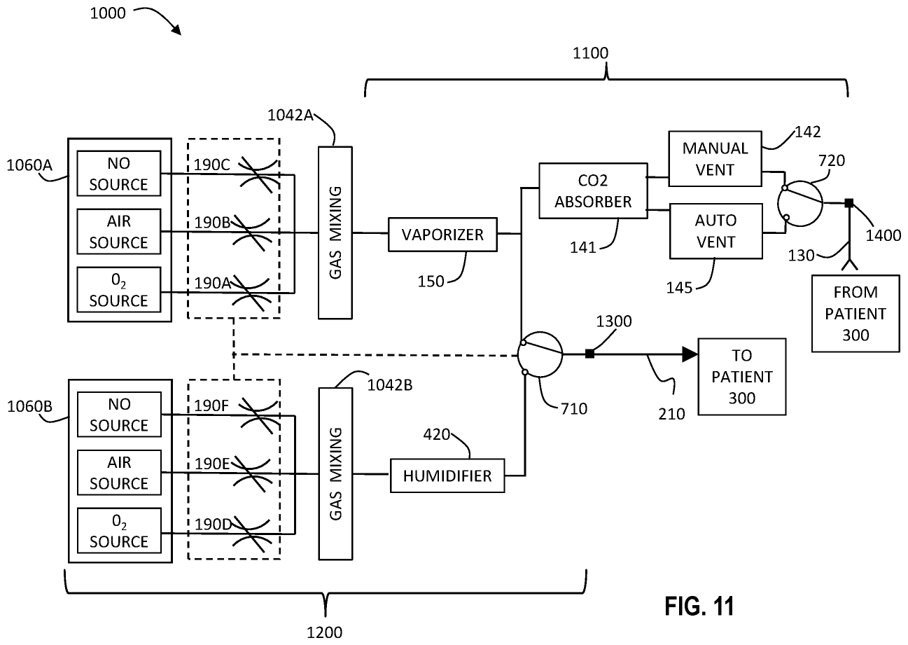

FIG. 11 provides a variation of the embodiment in FIG. 10, which provides separate sets of flow meters 190A, 190B, 190C and 190D, 190E, 190F and separate gas mixing elements 1042A, 1042B. The first set of flow meters 190A, 190B, 190C control the flow of each of O2, Air and NO into a first gas mixing element 1042A from which the first gas flow path 1100 receive gases. The second set of flow meters 190D, 190E, 190F control the flow of each of O2, Air and NO into a second gas mixing element 1042B from which the second gas flow path 1200 receive gases. Although FIG. 11 shows separate gas sources 1060A and 1060B, this is for ease of representation of flows in the figures. It is to be understood that in the embodiment shown it is not necessary to have separate gas sources 1060A and 1060B supply each of the first and second gas flow paths 1100 and 1200. It is contemplated and expressly stated that each of the first and second gas flow paths 1100 and 1200 can receive gases into their respective sets of flow meters from a single source of each of one or more gases provided to the patient. Where first and second gas flow paths 1100 and 1200 receive gases from a single source it is to be understood that, in some embodiments, NO may not be supplied to the second gas flow path 1200.

Furthermore, while vaporizer 150 is shown downstream of gas mixing element 1042A, it is to be understood that the position of these elements may be reversed including in arrangements where the first and second gas flow paths 1100 and 1200 receive gases from a single source.

In FIG. 11, there is an operative coupling between each of the first set of flow meters 190A, 190B, 190C and the second set of flow meters 190D, 190E, 190F, and a switching mechanism comprising switching element 710 as shown by the broken lines. As described previously, the switching element 710 is operable to switch the flow of gas to the first gases outlet 1300 between the first gas flow path 1100 and the second gas flow path 1200. In the arrangement of FIG. 11, when the switching element 710 is operated to enable the first gas flow path 1100 (as in the illustrated configuration) the operative coupling between switching element 710 and the flow meters causes first set of flow meters 190A, 190B, 190C to operate to deliver gases at flow rates suitable for achieving respiratory support in the first mode of operation, and shuts off flow from the second set of flow meters 190D, 190E, 190F. Conversely, when the switching element 710 is operated to enable the second gas flow path 1200, the operative coupling between switching element 710 and the flow meters causes the second set of flow meters 190D, 190E, 190F to operate to deliver gases at flow rates suitable for achieving respiratory support in the second mode of operation, and shuts off flow from the first set of flow meters 190A, 190B, 190C. The required flow rates for each flow meter may be pre-set for operation to supply the designated gas flow path akin to the description of FIG. 10. For example: when the first gas flow path 1100 is enabled, flow meters 190A, 190B, 190C may be configured to deliver a flow rate for the mixed gas that is suitable for provision of respiratory support in the first mode of operation (e.g. 15 L/min at the first gases outlet 1300). When the second gas flow path 1200 is enabled, flow meters 190D, 190E, 190F may be configured to deliver a flow rate for the mixed gas that is suitable for provision of respiratory support in the second mode of operation (e.g. 40 L/min or 70 L/min at the first gases outlet 1300). In some embodiments, when the second gas flow path 1200 is enabled, the gas flow meters may be configured such that only O2 gas flow meter 190D provides a flow of gas to gas mixing element 1042B, with Air and NO flow meters 190E, 190F disabled or providing zero flow. In other cases, it may be desirable that one or both of gas flow meters 190DE, 190E provide flow to gas mixing element 1042B.

Alternatively/additionally, the flow meters 190A, 190B, 190C, 190D, 190E, 190F may be adjustable through manual operation or via a user interface 1094 in arrangements where the flow meters are in operative communication with a controller 1010.

The examples in FIGS. 6 and 7 provide a switching element 710 as part of the switching mechanism of the device 1000. The switching element 710 may comprise actuator, knob switch or an input interface 1094 operable by a user to select the mode of operation. Alternatively/additionally, the switching mechanism may comprise operation of a controller 1010 of the device 1000 to switch the mode of operation in response to a change in operating condition (such as application or removal of a sealing patient interface to/from the patient) as determined by sensors providing inputs to the controller.

Advantageously, the embodiments of FIGS. 4-7 remove the need to set up an additional oxygen supply since both the first gas flow path 1100 which enables operation of the device in a first mode of operation, and the second gas flow path 1200 which enables operation of the device in a second mode of operation, can receive oxygen from a common gases supply 1060. Furthermore, this arrangement facilitates simultaneous switching of both the first gas flow path 1100 and the second gas flow path 1200 such that when the second mode is selected, the first gas flow path 1100 ceases to deliver gases to the patient 300. This provides improved safety by preventing delivery of anaesthetic agents including NO and volatile anaesthetics vaporized in the first gas flow path 1100 in the flow of gases to the first gases port 1300. Furthermore, it does not allow anaesthetic agents to enter the environment avoiding inhalation of these agents by carers attending to the patient, while also reducing wastage.

In some embodiments comprising a controller 1010 and user interface 1094, the device may provide a prompt such as an audible, visible or tactile prompt to the user when switching to enable the second gas flow path 1200, prompting the user to continue to use the first patient interface 120 on the patient until there are very low amounts of or no anaesthetic agents in expired gases returned to the device at second gases port 1400. Such an alert provides a safety feature to reduce or remove the risk of anaesthetic agents being leaked into the care environment. Alternatively/additionally, the controller 1010 may be configured to provide a prompt (e.g. second prompt) prompting the user that it is safe to remove the first patient interface 120 from the patient. The controller 1010 may be configured with a timer and the second prompt given when sufficient time has elapsed after switching that there are very low amounts of or no anaesthetic agents in expired gases returned to the patient. In some examples, user interface 1094 may provide a visible and/or audible countdown timer providing the user with a guide as to when the elapsed time will end indicating that that there are very low amounts of or no anaesthetic agents in expired gases and the first patient interface 120 may be removed. Alternatively or additionally the device 1000 may comprise one or more sensors in the expired gases flow path to detect the presence of anaesthetic gases, and the controller 1010 may be configured to provide the second prompt only when the sensors indicate that no anaesthetic gases are detected or that the amount of anaesthetic gases detected are below a predetermined safety threshold.

An electronically enabled switching element 710 may be physically attachable to one or both of the first or second patient interfaces 120, 220 to enable quick switching between gas flow paths for operation of the device 1000 in different modes when the patient interface is swapped, e.g. when moving from pre-intubation to intubation, or when weaning the patient from sedation. For example, switching element 710 may comprise a button or electronic switch located on the patient interface which may be actuated, causing device 1000 to switch to the mode of operation that will safely deliver gases to the patient through that interface. Alternatively a foot pedal or foot-operated switch or voice control may be utilised. Each of these has the potential to improve accessibility of gas flow path/mode selection while the user is physically distant from the controls located on the device itself.

While some embodiments of the disclosure provide for switching between enabled gas flow paths (and modes of operation) of the device 1000 based on a user input provided manually to a switching element or through a user interface, in some embodiments device 1000 comprises a controller 1010 receiving inputs from one or more sensors that are configured to detect a condition of operation of the device, such as if the first patient interface 120 is applied to the patient to receive expired gases, since this is appropriate only for use when the first gas flow path 1100 is enabled. Thus, when the sensor/s detect that the first patient interface 120 is applied to the patient then the controller 1010 ensures the first gas flow path 1100 is enabled such that the device is configured for operation in the first mode (anaesthesia rebreathing) or the third mode (ventilation rebreathing) which may be considered a specific example of the first mode, and which collectively or individually may be regarded as a “rebreathing” mode. Conversely, when the sensor/s detect that the first patient interface 120 is not applied to the patient then the controller 1010 ensures the second gas flow path 1200 is enabled such that the device is configured for operation in the second mode delivering Nasal High Flow (NHF) respiratory support.

A range of different approaches and combinations of approaches to sensing may be used as a means to provide gas flow path and/or mode switching in a device 1000 for delivering gases to a patient. In one example, pressure in the mask cavity may be monitored by a pressure sensor and used to detect when the mask is in place on the patient. Detection of the mask on the patient may be achieved by detecting an increase in mask pressure from pressures expected during delivery of high flow respiratory support (consistent with a mask being placed over the cannula) and causes the switching mechanism to disable the second gas flow path 1200 and enable the first flow path 1100 such that configuration of the device is switched from the second (high flow) mode of operation to the first (rebreathing) mode. Additionally or alternatively, an optical sensor may be provided in the mask which is configured to monitor changes in emitted/detected light that occurs in the presence of the patient's skin, and may be used to detect placement of the mask on the patient. In some embodiments this may be used in conjunction with a pressure sensor (e.g. to sense pressure in the cuff of a mask or in the mask cavity) which detects an increase in pressure when the mask is applied to the patient which cause the switching mechanism to disable the second flow path and enable the first flow path such that there is a switch from the second mode to the first mode of operation. In some embodiments, a pressure sensor in the cuff of a mask may be used independently of an optical sensor. Likewise, if an opposite change in these measurements is detected then this may be consistent with the mask being removed from the patient which may cause the switching mechanism to disable the first flow path and enable the second flow path such that there is a switch from the first mode to the second mode of operation. Additionally or alternatively, one or more pressure sensors may be provided on an outside surface of a nasal cannula. For example, a pressure sensor may be provided at a region of the cannula over which part of a mask cuff is applied for operation of the device in the first mode, and/or at a region of the cannula which is located inside the sealing face mask when it is applied to the patient. An increase in cannula pressure signifies application of the mask over the cannula and causes the switching mechanism to disable the second flow path 1200 and enable the first flow path 1100 for operation in the first (rebreathing) mode.

Alternatively or additionally the sensors may include a pressure sensor arranged to measure pressure in one or both of the first gas flow path 1100 and the second gas flow path 1200. The device 1000 may provide a continual or intermittent flow of gases through the first gas flow path 1100 and/or the second gas flow path 1200 to enable a measurement of the pressure in the device. In some embodiments, this continual or intermittent flow of gases comprises a flow rate, pressure and/or volume less than the flow of gases provided to the patient in the first and/or second mode. Different resistance to flow values are associated with each of the first (sealing) patient interface 120 and second (non-sealing) patient interface 220 when coupled to the patient 300 which are used in the first and second modes respectively. When the measured pressure indicates expired gas is returned from the patient by a sealing patient interface that is substantially sealed with the patient, the controller 1010 causes the switching mechanism to enable the first gas flow path 1100 and operates the device 1000 in the first mode in which gases which may include anaesthetic agents are delivered to the patient 300, and expired gases are returned to the rebreathing components 140 of the first gas flow path 1100. Alternatively, when the measured pressure indicates breathing gas is delivered to the patient by a non-sealing patient interface (i.e. with no mask present to return expired gases), the controller 1010 causes the switching mechanism to enable the second gas flow path, and operates the device 1000 in the second mode in which gases excluding anaesthetic agents are delivered to the patient at the desired (high flow) flow rate. In some embodiments, the pressure sensor may be located at or downstream of a flow modulator 250 of the device 1000, or at any convenient location in the gas flow pathway between the patient airway and the flow modulator 250.

Alternatively or additionally, the one or more sensors may include a CO₂ sensor associated with an inspiratory gas flow path (such as in one or more of first gases port 1300, first conduit 210, first patient interface 120 and/or second patient interface 220 depending on the interface configuration) and/or an expiratory gas flow path (such as in one or more of second gases port 1400, second conduit 130 and first patient interface 120). In such embodiments, the controller 1010 determines the gas flow path containing a higher concentration of CO₂ than ambient air is associated with the patient interface that is coupled with the patient's airway. If CO₂ concentration in both flow paths is higher than ambient air, the controller determines the flow path with the higher CO₂ concentration is associated with the patient interface that is receiving expiratory gas and so is coupled with the patient's airway, and causes the device 1000 to enable the first gas flow path 1100 such that the device is operational in the first (or third) mode, with the second gas flow path 1200 disabled.

Alternatively or additionally the one or more sensors may comprise an O2 sensor measuring a characteristic (e.g. gas concentration) of O2 in the expiratory flow path. Knowing the characteristics of the O2 supplied to the patient during respiratory support, the controller 1010 can compare the sensed O2 characteristic with the supplied O2 characteristic and determine, when it is equal or close to or within a range of the supplied value, that the first patient interface 120 is applied to the patient. Alternatively, if the sensed O2 characteristic (e.g. concentration) is greater than atmospheric O2 concentration (e.g. greater than 22% or greater than 25%) the controller 1010 can determine that the first patient interface is applied to the patient. Alternatively/additionally, a trace gas (e.g. nitrous oxide or another inert gas) or a trace gas flow pattern (e.g. containing flow rate or pressure oscillations of known frequency or behaviour) may be provided in addition to the respiratory support in the first conduit 210. If the controller determines the trace gas or trace gas flow pattern to be present in the expiratory gases it can determine that the first patient interface is applied to the patient.

Alternatively or additionally, sensing in the expiratory flow path may be utilised to determine when the first patient interface 120 (e.g. sealing mask) is in place on the patient to trigger the switch to the first (rebreathing) mode. One or more parameters in the expiratory flow path such as flow rate, pressure, temperature, or humidity may be determined using suitable sensors. An increase in one or more of these parameters (e.g. if a sensor determines temperature in the expiratory flow path to have increased or to be higher than ambient), would indicate the mask is on the patient and trigger switching to enable the first gas flow path configuring the device 1000 for operation in the first (or third) mode.

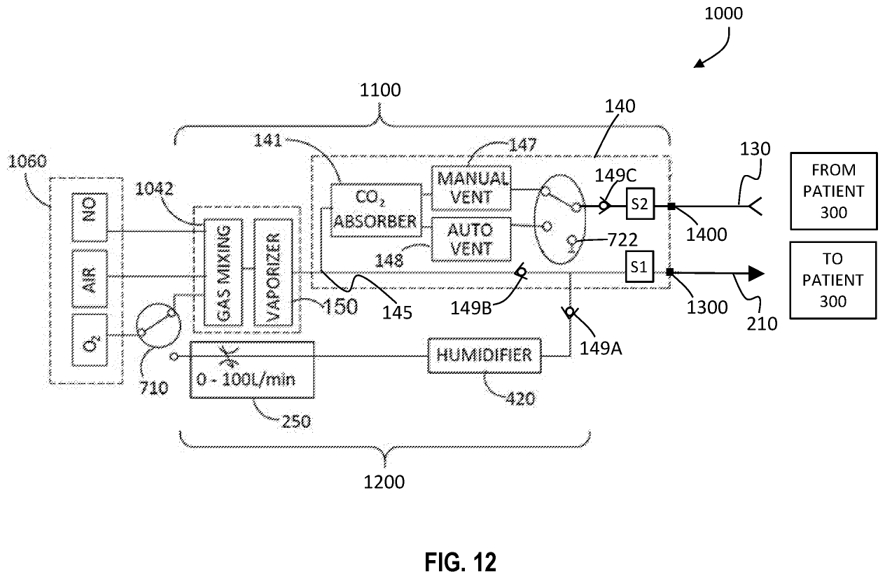

FIG. 12 is a schematic illustration showing two example locations, S1 and S2, for sensors for detecting characteristics of gases in the inspiratory flow path 210 and expiratory flow path 130 respectively. Sensors may be provided at one or both locations S1, S2 for determining e.g. if the first patient interface 120 is applied to the patient (or not). Sensors at S1 and/or S2 may sense characteristics of gases such as but not limited to pressure, flow rate and gas species concentration (e.g. O2, CO2 concentration). These sensors may be main stream sensors (with the sensing component located in the inspiratory or expiratory gas flow path) or side stream sensors which receive sampled gas tapped from the inspiratory or expiratory gas flow path. Each location S1, S2, may comprise zero, one or more than one sensor, and sensor types may be selected according to one or more characteristics of the gas to be sensed. Location S1 in the inspiratory flow path 210, and/or in the device 1000 between the junction 145 of flow paths connecting rebreathing components 140 and gases outlet port 1300, may be beneficial for sensor-driven determination of the enabled flow path since a single sensor at S1 can be used to measure flow in both the first gas flow path 1100 and the second gas flow path 1200. A gas pressure sensor at S1 may be used to determine whether or not the first patient interface is applied to the patient since pressure measured at S1 can be compared to an expected pressure value that corresponds to a mode of respiratory support being provided. In an example, a first mode may be a rebreathing mode with a first (sealing) patient interface applied to the patient, and a second mode may be a high flow mode with the first patient interface not applied to the patient.

Alternatively or additionally, a sensor may be provided at S2 to determine a characteristic of expired gases in the expiratory conduit 130. A sensor provided at S2 may be used to determine whether or not a first (sealing) patient interface 120 has been applied to the patient. In one example, when the device is operating in the second mode, detection of gas flows at S2 may be used to trigger switching to a first (rebreathing) mode of operation. Conversely, when the device is operating in the first mode, detection of an absence of gas flows at S2 may be used to trigger switching to the second mode of operation. Using more than one sensor, optionally at both sensor locations S1 and S2 provides redundancy in sensor driven automated switching between modes of operation of the device. This may mitigate false triggering of mode switching which could be caused by e.g. accidental flows from room ventilation or other sources. Additionally inaccurate performance or failure of one sensor alone will not compromise safe operation of the device when multiple sensors are provided to provide inputs to controller 1010 for determining when mode switching is to occur.

It is to be understood that sensor locations S1 and S2 in FIG. 12 are examples only. Flow rate sensors sensing flows in the second gas flow path 1200 may be located anywhere downstream of flow modulator 250 or flow meters 190. For sensing flows in a first mode of operation, flow rate sensors may be located anywhere downstream of gas mixer 1042. For sensing flows in either mode of operation, flow rate sensors may be located anywhere downstream of non-return valves 149A,B, and/or anywhere downstream of first gases port 1300. Alternatively or additionally, flow rate sensors sensing flows in expiratory flow path 130 may be located anywhere in the flow path between the patient 300 and the junction 145. This may include a location between patient 300 and second gases port 1400 and/or a location inside device 1000 between second gases port 1400 and junction 145, and/or between second gases port 1400 and the non-return valve 149C. Pressure sensors may be positioned at the same location as flow rate sensors, in addition to or as an alternative to flow rate sensors.

O2 sensors sensing O2 concentration in the inspiratory flow path 210 may be located anywhere downstream of flow source 1060 in either mode of operation, including downstream of non-return valves 149A, B and/or downstream of first gases port 1300. Alternatively or additionally, O2 sensors sensing O2 concentration in the expiratory flow path 130 may be located anywhere in the flow path between the patient 300 and the junction 145. This may include a location between patient 300 and second gases port 1400 and/or a location inside device 1000 between second gases port 1400 and junction 145, and/or between second gases port 1400 and the non-return valve 149C. CO2 sensors sensing flows in the expiratory flow path 130 may be provided anywhere in the flow path between the patient 300 and the CO2 absorber 141 when provided. This may include a location between patient 300 and second gases port 1400 and/or a location inside device 1000 between second gases port 1400 and non-return valve 149C, and/or between non-return valve 149C and CO2 absorber 141.

In some examples, it may be beneficial to locate sensors on the device side of the first and/or second gases ports 1300, 1400 to avoid the sensors being incorporated into consumable/disposable components of the system, thereby reducing costs. In some examples, however, it may be beneficial to sense characteristics of the gases closer to the patient, e.g. within the patient interface or within the conduit coupled with the patient interface for accuracy and to mitigate false readings (and potential mode switching) arising from accidental crushing of conduit or other movement that could impact sensor measurements.

Sensor driven automated switching between gas flow paths for operation of the device 1000 in different modes may be achieved by the controller 1010 receiving sensor inputs and based on those inputs, controlling operation of switching elements of the device when certain pre-defined conditions are met. In an example, one or more sensors which may sense one or more of pressure, flow rate and gas concentration in the expiratory flow path 130 provide inputs to controller 1010. If the flow rate or pressure or gas concentration meets a predetermined condition e.g. is greater than a predetermined threshold e.g. flow rate is greater than 0 or about 0.5 to about 2 L/min, and/or pressure greater than ambient air, and/or CO2 concentration greater than ambient air (if the patient is breathing) and/or O2 concentration is greater than ambient air (if O2 provided to patient is greater than 21%), then the controller determines that the sealing mask is applied to the patient and the system is operable in the first (rebreathing) mode.

Conversely, if the sensed parameter meets a predetermined condition e.g. is below a predetermined threshold, the controller determines that the sealing mask is not applied to the patient and the system is operable in the second (high flow) mode.

In some examples sensing a gas concentration parameter, the predetermined condition may comprise the sensed gas concentration being at or near the predetermined threshold, if the threshold corresponds to gas concentration values corresponding to ambient air. If the device 100 is not already operating in the appropriate mode, the controller operates switching elements of the device to provide respiratory support in the appropriate mode. Thus, if the device is providing respiratory support in the first mode and the controller determines the sensor input/s to be below the threshold, the control is switched to provide respiratory support in the second mode. Conversely, if the device is providing respiratory support in the second mode and the controller determines the sensor input/s to be above the threshold, the control is switched to provide respiratory support in the first mode. It may be desirable to set the threshold for CO2 and O2 concentration to be just above ambient values to mitigate false threshold detection due to environmental flows e.g. from room ventilation or other sources. The thresholds utilised by the controller 1010 in sensor driven automated switching may be adjustable e.g. by a user or service technician. In some examples, the threshold that triggers switching from the first mode to the second mode may be the same as the threshold that triggers switching from the second mode to the first mode. Alternatively, these switching conditions may be triggered by different threshold values to allow for tolerance and certainty of switching and/or to mitigate e.g. undesirable oscillations between modes when a single threshold is used.

In another example, one or more sensors may sense pressure in inspiratory flow path 210. While the device is operating in the second mode, controller 1010 may receive continuous or regular inputs from the pressure sensor/s and compares these with a threshold or range of high flow pressure values known to be consistent with provision of respiratory support in the second (high flow) mode. When the controller detects an increase in pressure above the threshold or range of high flow pressure values, the controller determines that the sealing mask is applied to the patient and controls the switching elements of the device to provide respiratory support in the first mode. Similarly, while the device is operating in the first mode, controller 1010 may receive continuous or regular inputs from the pressure sensor/s and compares these with a threshold or range of rebreathing pressure values known to be consistent with provision of respiratory support in the first mode. When the controller detects a decrease in pressure below the threshold or range of rebreathing pressure values, the controller determines that the sealing mask is not applied to the patient, and controls the switching elements of the device to provide respiratory support in the second mode.

In another example, one or more sensors may sense pressure and/or flow rate and/or gas concentration in the expiratory path 130, and provide inputs to controller 1010. If the controller 1010 determines that a time averaged flow rate or O2 concentration of gases in the expiratory flow path 130 is about equal to, or close to, or within a percentage (e.g. within 90+%) of a flow rate or O2 concentration provided as part of the respiratory support the controller determines that the mask is applied to the patient and, if the respiratory support is in the second mode, controls the switching elements of the device to switch the respiratory support to the first mode. Conversely, if the controller 1010 determines that a time averaged flow rate or O2 concentration of gases in the expiratory flow path 130 is not equal or close to or within a percentage (e.g. within 90%) of a flow rate or O2 concentration provided as part of the respiratory support the controller determines that the mask is not applied to the patient and, if the respiratory support is in the first mode, controls the switching elements of the device to switch respiratory support to the second mode. In some examples, one or more sensors may also sense pressure and/or flow rate and/or gas concentration in the inspiratory path 210 to determine the characteristics of the respiratory support provided to the patient. It is relevant to note that the characteristics, such as the flow rate, O2 concentration and gas flow pressure provided as part of the respiratory support will differ according to the mode of respiratory support being provided. It may be preferable for the values of pressure and/or flow rate and/or gas concentration to be time averaged over at least a breathing cycle (if the patient is spontaneously breathing) to allow for measurement delay.

Alternatively or additionally, the respiratory support provided to the patient may comprise a controlled component providing a “signature” in the gases flow that can be used by the controller to ascertain if the mask is or is not applied to the patient. A signature may comprise e.g. an oscillation in a feature such the frequency, amplitude or profile of the pressure and/or flow rate and/or O2 concentration of the gases provided to the patient. Oscillations may be achieved e.g. by a valve such as a proportional valve in the flow path. During provision of respiratory support, if the controller determines that the signature is present in gases received in the expiratory flow path 130 the controller determines that the mask is applied to the patient and, if the respiratory support is in the second mode, controls the switching elements of the device to switch the respiratory support to the first mode. Conversely, if the controller determines that the signature is not present in gases received in the expiratory flow path 130 the controller determines that the mask is not applied to the patient and, if the respiratory support is in the first mode, controls the switching elements of the device to switch the respiratory support to the second mode. In some embodiments, the signature may be provided in only one mode, or different signatures may be provided for different modes of respiratory support.

In examples where the controller 1010 compares sensed flow rate values with reference or threshold values, the controller may calculate a difference as an absolute value and/or a percentage of threshold or provided respiratory support. Controller 1010 may be further configured to display the calculated difference on the user interface 1094. In the case of the controller determining a difference between provided values and sensed values, the display of the difference may provide useful data which could indicate presence of a leak in the system. When a sealing mask is being used in the first mode, this display could indicate insufficient sealing of the mask over the patient's face.

It is to be understood that the controller 1010 may utilise and/or combine any one or more of the sensing examples in the foregoing to provide greater certainty that the conditions for switching are met thereby reducing the likelihood of false detections causing erroneous switching. By way of example only, one such combination may comprise flow rate plus CO2 detection in the expiratory flow path 130, or flow rate detection in the inspiratory flow path 210 plus gas concentration sensing in the expiratory flow path 130. Another combination may comprise pressure sensing in the inspiratory flow path 210 plus pressure sensing in the expiratory flow path 130 with a further optional addition of CO2 sensing in the expiratory flow path 130.

Any one or more of e.g. pressure, flow rate or gas concentration sensing in the expiratory flow path 130; pressure sensing in the inspiratory flow path 210, and pressure, flow rate and/or gas concentration sensing in the inspiratory or expiratory flow paths 210, 130 may be used by the controller to control switching from the second mode to the first mode, and the same or different one or more sensor inputs may be used by the controller to control switching from the first mode to the second mode. In some cases, it may be desirable to have greater redundancy or certainty (and thus require more conditions to be met before switching) when the controller switches from the second mode to the first mode due to the release of volatiles. However in some cases it may be desirable to have greater certainty when switching from the first mode to the second mode to ensure that it is safe and/or appropriate before stopping ventilation or provision of volatiles in the inspiratory gas flow path.

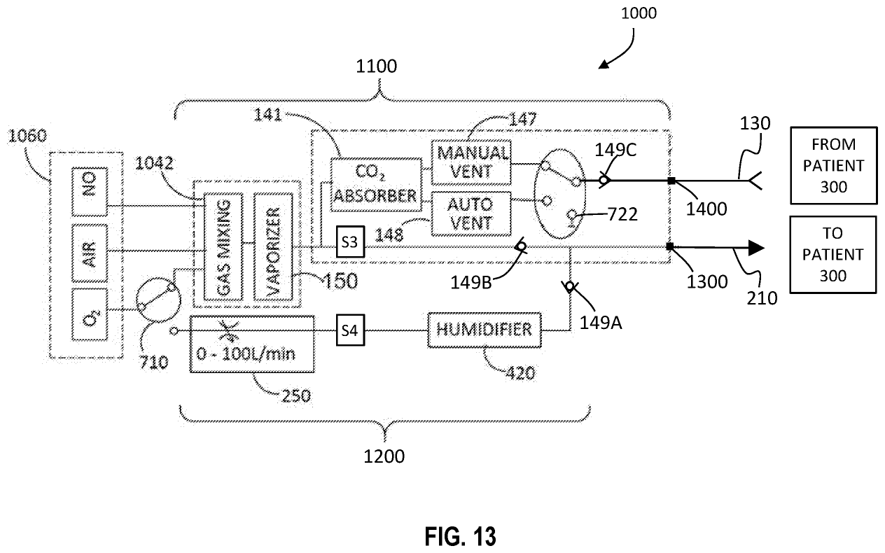

In another example shown schematically in FIG. 13, a small residual flow may be provided in the first and/or second flow path 1100, 1200 even when it is inactive (not enabled) and one or more sensors may be provided in locations S3 and/or S4 upstream of non-return valves 149. The residual flow may be in the order of e.g. about 0.5 L/min to about 5 L/min to ensure that sensors at locations S3 and/or S4 are in fluid communication with first gases port 1300 (for example, if there are non-return valves 149A, 149B in the flow path, the residual flow will keep these valves open). The residual flow may comprise air instead of O2 to reduce O2 consumption and/or wastage.

In an example, if the device is providing respiratory support in the second mode, a residual flow may be maintained through the rebreathing components 140 past S3 to keep the flow path open. Pressure at S3 may be regularly/continuously sensed and controller 1010 receives inputs from the pressure sensor/s and compares these with a threshold or range of values known to be consistent with provision of respiratory support in the second mode at a particular flow rate. When an increase in pressure is detected at S3 the controller determines the mask to be applied to the patient and controls the switching elements of the device to provide respiratory support in the first mode.

While operating in the first mode, a residual flow maintained in the second gas flow path 1200 keeps the flow path open. Pressure at S4 may be regularly/continuously sensed and controller 1010 receives inputs from the pressure sensor/s and compares these with a threshold or range of values known to be consistent with provision of respiratory support in the first mode. When a decrease in pressure is detected at S4, the controller determines that the mask is not applied to the patient, and controls the switching elements of the device to provide respiratory support in the second mode, at a flow rate higher than the residual flow rate. The controller may determine an increase or decrease in pressure at S3 and/or S4 by reference to a threshold value or range or values which may be absolute values (e.g. 1 cmH2) or a percentage of the expected pressure for a particular mode of operation (e.g. 20+% change from expected).

In the figures, non-return valves 149A,B are shown as separate valves upstream of the junction between the inspiratory flows from the first gases flow path 1100 and the second gases flow path 1200. However, the functionality of these valves may be combined into a single non-return valve provided downstream of the junction between the inspiratory flows from the first gases flow path 1100 and the second gases flow path 1200. Alternatively, a valve that prevents reverse flow could additionally be placed at this location downstream of the junction. In such an arrangement, a valve that prevents reverse flow may be desirable e.g. downstream or upstream of the humidifier 420 and/or at switching element 710 to prevent return flows exiting to atmosphere in the first (rebreathing) mode.

Alternatively or additionally, the sensors may include one or more proximity sensors such as e.g. acoustic (including audible and/or ultrasonic), optical (including infra-red), radiofrequency, pressure (in the inspiratory/expiratory conduits and/or mask cuff), flow, electrical conductivity, resistance, temperature or other sensors to determine which of the breathing circuits is coupled with the patient's airway by the first or the second patient interface. Such proximity sensors are explained in further detail in WO2016157105A1, the contents of which are hereby incorporated herein by reference.

In some examples of automated (sensor driven) switching between modes of respiratory support, the controller 1010 may apply a timing control to the switching control, wherein upon detection of a condition that should trigger a mode switching, the controller applies a delay before controlling the switching elements to cause the mode switch. This may avoid erroneous switching arising from sensing errors or transient conditions due to e.g. accidental crushing of conduit or other movement that are resolved after a few seconds. The user interface 1094 may provide a visible and/or audible countdown timer providing the user with an indication of when switching between modes will occur. It may also be desirable for the user interface 1094 to provide an option for a user to cancel a mode switching proposed by the controller within the predetermined time, e.g. if the user is aware there has been an erroneous switching condition met (a false trigger). For example, when switching from the first mode to the second mode, the controller may control operation of one or more of gas source 1060, 1600A, 1060B and flow meters 190C, 190F to cease supply and/or reduce the flow rate of volatiles being provided in the first gas flow path 1100 but the therapy through the first gas flow path may otherwise continue until the predetermined time has elapsed. If the proposed switching is cancelled by the user during the predetermined time period, the controller will resume provision of volatiles and there will be no cessation of flow from the first gas flow path 1100 to the patient.

The switching mechanism 1370 may comprise one more actuators that are operable by a user, such as, but not limited to one or more of a button, switch, knob, foot operated switch or pedal. Alternatively or additionally switching mechanism 1370 may comprise or be in operable communication with one or more of an electronic input device, touch screen, voice activated sensor or the like, as may be operable in concert with an electronic controller 1010 of the device 1000. One or more actuators of the switching mechanism 1370 may be located at or near a first or second patient interface 120, 220 through which expired gas removed or breathing gas is delivered to the patient. Locating one or more of the actuators at or near the patient end of a breathing circuit delivering gases to the patient's airway provides convenience for a clinician working on the patient throughout phases of anaesthesia where it is often necessary to switch between modes of operation of the device 1000 and the form of respiratory support that is delivered. Locating an actuator enabling mode selection at the patient end may be more convenient and time saving for the clinician and others in their vicinity. In other arrangements, the device 1000 may be configurable to detect if the first patient interface, being an expiratory patient interface configured to receive expired gases from the patient for return to the device, is attached to the patient, and modify the mode of operation and flow path selection accordingly.

In some embodiments, user interface 1094 may provide an audible and/or visible output (such as an indication on a display screen and/or audible sound or message) to indicate to the user the mode of operation that the device 1000 is operating in. Alternatively or additionally, another component of the respiratory support system such as a gas conduit or patient interface of the breathing circuit may comprise an audible or visible output element (such as a speaker and/or LED or other illuminating element) operable by controller 1010 to provide an output to indicate to the user the mode of operation that the device 1000 is operating in. In an example, a predetermined colour and/or sound and/or symbol on the output element may be associated with operation of the device 1000 in the first mode, whereas a different predetermined colour and/or sound and/or symbol may be associated with operation in the second mode. The one or more predetermined colours and/or sounds and/or symbols may be user selectable and/or user defined. Locating one or more of output elements at or near the patient end of a breathing circuit delivering gases to the patient's airway provides convenience for a clinician working on the patient to remain apprised of the mode in which the device is operating throughout phases of anaesthesia.

It is to be understood that the switching mechanism 1370 may include one or more switching elements such as mechanical, electronic, electromechanical, electromagnetic, pneumatic or any other suitable switching mechanisms to achieve the functionality disclosed herein. Furthermore, the one or more switching elements may be couplable with the switching mechanism 1370 via wired and/or wireless coupling using techniques readily understood and ascertainable by one of skill in the art. Switching elements may include actuators operable by a user of the device 1000 to select the required mode of operation (and flow path to be enabled) and may include or consist of any of the switching elements described. In some embodiments, a switching element may comprise an electronic input device in wireless communication with a controller 1010 of the device 1000 and movably locatable to different positions with respect to the patient 300 and/or the device 1000 and/or the patient interfaces 120/220.

In some embodiments, the one or more switching elements of the switching mechanism 1370 may be operable to control one or more characteristics of gases delivered to the subject, such as but not limited to presence of volatiles, flow rate, gas composition, gas concentration, temperature, and/or humidity.

For conciseness, certain features of the first gas flow path 1100 are not always shown in the figures. However it is to be understood that in preferred embodiments the first gas flow path 1100 performs the function of an anaesthesia machine 10 and/or a ventilator 20 and comprises one or more of a CO₂ absorber 141 configured to treat returned expired gas before recirculating to the patient in the first mode, a pressure limiting valve 146 configured to maintain substantially stable pressure in the device in the first mode, a variable volume 145 for displacement of gases in the first mode (e.g. using bellows or a bag ventilator), a replenishing gas flow for replenishing anaesthetic gas delivered to the patient in the first mode and a vaporizer 150 for vaporizing volatile anaesthetic agents into gas delivered to the patient in the first mode.

Similarly, for conciseness certain features of the second gas flow path 1200 are not always shown in the figures. However it is to be understood that in preferred embodiments the second gas flow path 1200 delivers high flow respiratory support as described herein, and comprises one or more of a flow source 250 or modulator configured to generate gas flows through the second gas flow path 1200 in the second mode. A humidifier 420 configured to condition the gas to a pre-determined temperature and/or humidity before delivery to the first gases port 1300 may also be provided although in some cases it may be omitted.

In some embodiments, a switching mechanism may be provided by simultaneous use of the first and second patient interfaces 120, 220. Thus when the first (expiratory) and second (inspiratory) patient interfaces are applied to the patient simultaneously the first gas flow path 1100 is enabled, configuring the device 1000 for operation in the first mode, and when only the second (inspiratory) patient interface is applied to the patient the second gas flow path 1200 is enabled configuring the device 1000 for operation in the second mode. In one arrangement, the first patient interface 120 is a mask which is capable of sealing over the second patient interface 220 being a nasal cannula, without occluding flow through the cannula. Inspiratory flow is delivered via the cannula with expiratory gases returned via the face mask. This arrangement can be deployed to deliver anaesthesia in a closed system utilising the cannula to deliver anaesthetic agents to the patient and the mask to return expired gases. Pressure sensors may be used to determine when the mask is applied to the patient and sealed over the cannula, such that when a target pressure is detected at the mask (e.g. inside the mask or within the mask cuff, or with pressure sensors on the cannula body), then the switching mechanism 1370 is triggered thereby enabling the first gas flow path 1100 provision of anaesthetic agent through the nasal cannula. Commonly owned patent publication WO2015145390 discloses a mask suitable in this context and is hereby incorporated herein by this reference.

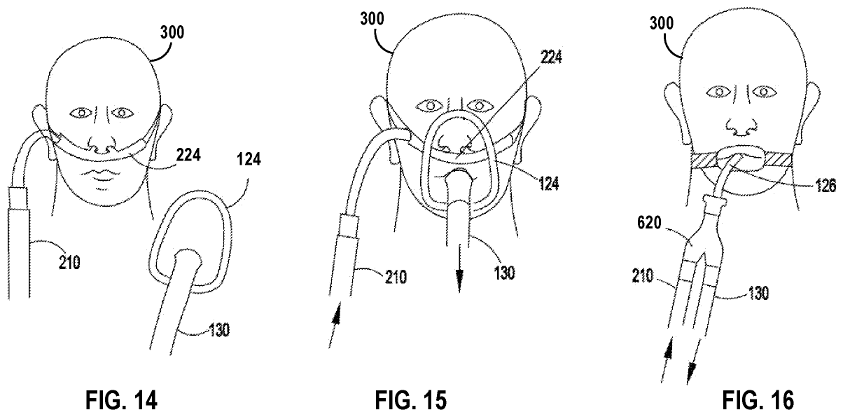

FIGS. 14 to 16 illustrate one example of how switching between flow paths within the device 1000 as disclosed herein to configure the device for use in different modes of respiratory support may be achieved by simultaneous or separate use of the first and second patient interfaces 120, 220. The device 1000 provides a first gases port 1300 configured for coupling with a first (inspiratory) conduit 210. A second gases port 1400 is provided for coupling with a second (expiratory) conduit 130 to return expired gases from the patient via first patient interface 120. When either flow path of device 1000 is enabled, gases are provided via the conduit 210 and a second patient interface 220 to the patient's airway. When the first flow path 1100 is enabled, the device 1000 is operable in the first mode to provide gases to a patient using a first flow parameter, and to return expired gases from the patient to the second gases port 1400 via the first patient interface 120 and second conduit 130. When the second gas flow path 1200 is enabled the device 1000 is operable in the second mode to provide gases to a patient using a second flow parameter. The first and second flow parameters include or correspond to a first and second flow rate respectively. In some embodiments, the first flow rate is less than 15 L/min and the second flow rate is greater than 15 L/min. In some embodiments, the second flow rate is in the range of between about 20 L/min and about 90 L/min, optionally between about 40 L/min and about 70 L/min. It is to be understood, however, that the first flow parameter may alternatively or additionally include a pressure and/or volume parameter.

As shown in FIG. 14, the second patient interface 220 is a non-sealing patient interface shown as nasal cannula 224 which is in fluid communication with first conduit 210, and the first patient interface is a sealing patient interface shown as mask 124. In FIG. 15, the nasal cannula 224 and mask 124 are applied to the patient simultaneously such that the device 1000 is operable in the first mode providing anaesthetic ventilation, with the mask 124 configured to seal over the nasal cannula 224 and with the patient 300. In this arrangement, gases are delivered to the patient via the first conduit 210 and expiratory gases from the patient are returned to the device 1000 from the patient via the mask 124 and the second conduit 130. In FIG. 14, only the non-sealing patient interface, e.g. nasal cannula 224, is applied to the patient for delivery of gases to the patient's airway in the second mode providing high flow respiratory support. In some embodiments, the second conduit 130 is not required or is inoperable in the second mode.

In an alternative embodiment, the gases may be delivered to the patient via second conduit 130 and the mask 124, and expiratory gases from the patient are returned to the device 1000 from the patient via the nasal cannula 224 and first conduit 210. In such an embodiment, the second conduit 130 is an inspiratory conduit and the first conduit 210 is an expiratory conduit.

Advantageously, physical application of the sealing mask 124 over the non-sealing cannula 224 as exemplified in FIG. 14 utilises the patient's own airway to provide a gas flow path between the cannula and the mask. This enables respiratory gases to be provided via the inspiratory conduit, and expired gases to be removed via the expiratory conduit without use of a wye-piece connector as is commonly used in re-breathing patient circuits to couple the inspiratory and expiratory limbs. Moreover, removal of the mask 124 leaves the cannula 224 in situ, ready for provision of respiratory support in the second mode of operation, without the requirement to apply a further patient interface to the patient. The reduction of components arising from elimination of a wye-piece connector, and simplification of the components and patient intervention steps can provide cost and time savings and simplify aspects of anaesthesia procedures.

FIG. 16 provides an arrangement for operation of the device 1000 in a third mode. In the third mode, ventilation rebreathing is delivered by an endotracheal tube 126. Here, the first conduit 210 has been decoupled from nasal cannula 224 and coupled with an inlet of coupling 620. Similarly, the second conduit 130 has been decoupled from mask 124 and coupled with an outlet of coupling 620. Patient end of coupling 620 has been connected with an endotracheal tube 126 providing the function of both the first and second patient interfaces in the third mode. In some embodiments the endotracheal tube 126 may be another type of patient interface such as a laryngeal mask airway or a tracheostomy interface or a mask. In the third mode, gases may be delivered to the patient including a third flow parameter which may include one or more of a flow rate, pressure or volume parameter. Although not shown in the Figure, it is to be understood that during operation of the device in the third mode using an endotracheal tube, laryngeal mask airway or tracheostomy interface, the second patient interface 220 comprising the nasal cannula 224 may remain in situ, on the patient, but disconnected from the first conduit 210. This may be beneficial in that it enables the user to easily switch between the third and first modes and/or the third and second modes of respiratory support.

An advantage of mode switching according to the examples described in relation to FIGS. 14 to 16 is that a single inspiratory conduit and a single expiratory conduit can be used to provide several modes of respiratory support to the patient. This reduces cost and complexity of breathing circuits utilised to treat the patient.

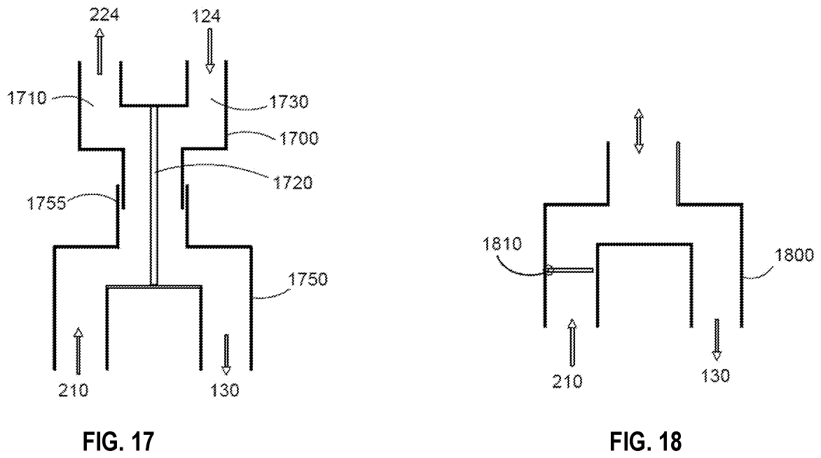

FIG. 17 is a schematic illustration of a novel connector 1700 that may be used to facilitate interchanging of components for delivery of respiratory support in the first and second modes as described in relation to the embodiments of FIGS. 14 to 16. The connector 1700 is configured to couple with a standard wye-piece connector 1750. In normal use, wye-piece connector 1750 is configured to couple at port 1755 with a conduit providing a flow of the respiratory gas to a mask 124 of the type shown in FIGS. 14 and 15. Wye-piece connector 1750 receives a flow of gas from first conduit 210 and provides an outflow pathway for expired gases from the patient via second conduit 130. When used consistent with FIG. 15, the mask 124 forms a sealed interface with the patient's airway for delivery of gases which may include anaesthetic agents via the nasal cannula 224, while returning expired gases to the device 1000 via the mask 124 and second conduit 130.

To facilitate interoperability between different patient interfaces, the novel connector 1700 may be used, which provides a wall 1720 configured to protrude into the wye-piece connector 1750. When connected, the wall 1720 separates the flows in the inspiratory and expiratory flows into separate limbs 1710 and 1730. In use, connector 1700 is configured to couple first limb 1710 with a conduit attached to a nasal cannula 224 and is configured to couple second limb 1730 with a conduit attached to a face mask 124. Owing to the separating wall 1720, inspiratory flow to the cannula 224 (see FIGS. 14, 15) remains separate from the expiratory flow received from the mask 124 (when used in the configuration of FIG. 15). To switch swiftly to the patient interface required to deliver support in the third mode, the connector 1700 can be removed from the wye-piece connector 1750 which instead is coupled with an endotracheal tube 126. Advantageously, connector 1700 can be used to couple and decouple the wye-piece connector 1750 from the nasal cannula 224 and the mask 124 simultaneously and with fewer disconnections/reconnections of parts for swift connection of the endotracheal tube 126 and less scope for error. When switching back to the first mode of support with the mask 124 configured to seal over the nasal cannula 224 (as per FIG. 15), the endotracheal tube 126 is decoupled from wye-piece connector 1750 and the connector 1700 is reconnected, simultaneously connecting the first and second conduits 210, 130 to the cannula 224 and mask 124.

FIG. 18 is a schematic illustration of another connector 1800 provided for use in the arrangement illustrated in FIG. 15 in lieu of the standard wye-piece connector 1750. Using connector 1800, the mask 124 may be used to remove expired gas via second conduit 130. For operation in the first mode (FIG. 15), connector 1800 may remain in place connected between the mask 124 and the second conduit 130 but decoupled from the first conduit 210 which is instead coupled with the nasal cannula 224. One way valve 1810 is biased for inspiratory flow in first conduit 210 toward the mask 124, operating to prevent expiratory gases from the mask from exiting to atmosphere since there is no inspiratory conduit attached to the connector 1800 in this mode of operation. To operate in the third mode (FIG. 16), the first conduit 210 can be reconnected to the connector 1800 and the mask 124 can be decoupled and replaced with connection of the endotracheal tube 126. An advantage of this arrangement is that connector 1800 may be used in delivery of both modes of respiratory support shown in FIGS. 9 and 10 while avoiding the need to disconnect and reconnect the second conduit 130 required to remove gases as is the case with a standard wye-piece connector.

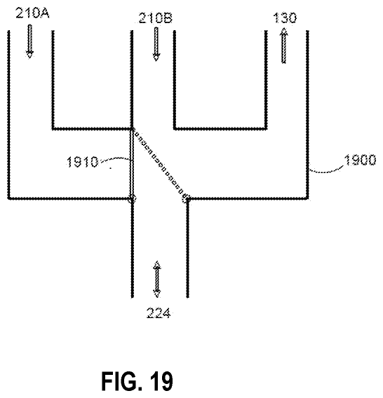

FIG. 19 is a schematic illustration of another novel connector 1900 configured for use in an embodiment where a nasal cannula 224 is used to deliver different modes of respiratory support. Connector 1900 is couplable with three conduits providing fluid communication with each of a first inspiratory gas flow path 210A configured to provide a flow of gases for delivery of nasal high flow respiratory support; a second inspiratory gas flow path 210B configured to provide a flow of gases for delivery of anaesthetic ventilation, and an expiratory gas flow path 130 for exit of expired gases from the patient. A switch 1910 (such as a switching valve, solenoid or the like) is provided to alter internal flow paths of the connector 1900 when a different mode of support is selected. When the nasal cannula 224 is used for delivery of anaesthetic rebreathing, switch 1910 is in the position shown in solid line, permitting delivery of respiratory gases (including anaesthetic agents) from the second inspiratory gas flow path 210B, and providing a pathway for expired gases, 130. During manual rebreathing, a bag mask can be applied over the nasal cannula 124 with sufficient pressure applied (e.g. by a clinical attendant) that the cannula can provide both the inspiratory and expiratory flow paths. In high flow respiratory support, switch 1910 is in the position shown in broken lines (diagonal position). Switch 1910 may be operatively linked with other switching elements in the device 1000 which are operated by a user (or a device controller 1010 with user interface 1094) to configure the device for operation in different modes of support.

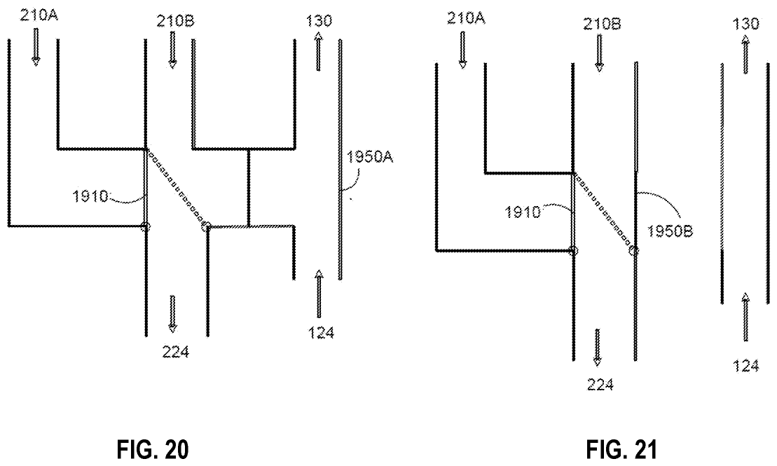

FIGS. 20 and 21 are schematic illustrations of a connector 1950A, 1950B which is a variation on connector 1900 from FIG. 19, wherein there is provision for connection with both a nasal cannula 224 and a mask 124. This connector 1950A, 1950B provides for delivery of nasal high flow when switch 1910 is in the broken line (diagonal) position. When switch 1910 is in the solid line (vertical) position anaesthetic rebreathing may be provided by cannula 224 with expiratory gases removed by application of face mask 124, with the apparatus arranged as shown in FIG. 15. FIG. 20 shows connector 1950A with all flow paths in a unitary connector piece. FIG. 21 shows connector 1950B comprising the inspiratory flow paths with connection for the nasal cannula 124, and a separate conduit used to provide the second (expiratory) conduit 130 attached to mask 124.

A device 1000 with which the patient interfaces shown in FIGS. 14 to 16 may be used may include a controller 1010 in communication with the flow source/modulator 250, and one or more sensors and/or a user interface in communication with the controller to provide an input to the controller to control the flow source/modulator to provide the flow of gases in the first or second mode. The sensors and/or user interface may also be configured to provide an input to the controller to control the flow source/modulator to provide the flow of gases in the third mode.

It is to be understood the terms “first”, “second”, “third” are used solely as labels to designate features of the disclosure and they are not to be taken as descriptive. Therefore, reference to a “second” feature does not necessitate provision of a “first” such feature, and reference to a “third” feature does not necessitate provision of a “first” and a “second” such feature. For example, provision of a second patient interface (which may be a high flow patient interface) in the present disclosure does not also require provision of a first patient interface (which may be a rebreathing patient interface).

### Advantages

Embodiments of the present disclosure provide a device that can deliver both high flow respiratory support and a rebreathing respiratory support for delivery of anaesthesia and/or ventilation, and enables easy switching between these modes of respiratory support. That is, embodiments of the present disclosure allow a clinician to easily switch between deployment of high flow respiratory support and rebreathing respiratory support for inducing/ventilating the patient. This reduces the overall number of components, simplifying the working environment and making it easier for an anaesthetist to perform required tasks during procedures involving high flow therapies or respiratory support, in addition to administration of anaesthesia. This can be beneficial e.g. when preparing to intubate the patient, or when weaning the patient off sedation. This arrangement can conveniently place features for user control for delivering high flow respiratory support together with features for user control for delivering sedation or deeper anaesthesia. Embodiments disclosed herein can also simplify and reduce the instrumentation and conduits that occupy valuable space in the clinical environment.

When switching to a high flow mode from a rebreathing mode, for safety reasons it is desirable that the transition stops the delivery of anaesthetic gases. Uncontrolled delivery of O₂ outside of the first gas flow path (e.g. when the mask is removed from the patient) could pose a fire risk, and there could be wastage of anaesthetic gas, as well as unintentional release of anaesthetic gases to the operating environment which could not only contaminate the high flow respiratory support gases, but impact the performance of individuals in the operating environment. Embodiments of the present invention may address one or more of these problems.

Also described herein are various embodiments, apparatuses, connectors, assemblies, accessories, devices and the like for achieving switching between modes of respiratory support. Some of these provide the convenience of controlling mode selection and/or displaying mode of operation when the user is not situated at the “machine”. Some embodiments provide for automatic selection of modes of operation by monitoring characteristics of gases such as gas pressure, O2 and CO₂ concentration in expired gases. Not only do these features improve convenience and operability of a device that provides these modes of respiratory support, they also have the capability to improve patient safety.

It is to be understood that various modifications, additions and/or alternatives may be made to the parts previously described without departing from the ambit of the present invention as defined in the provisional claims appended hereto.

The invention may also be said broadly to consist in the parts, elements and features referred to or indicated in the specification of the application, individually or collectively, in any or all combinations of two or more of said parts, elements or features. Where, in the foregoing description reference has been made to integers or components having known equivalents thereof, those integers are herein incorporated as if individually set forth.

Where any or all of the terms “comprise”, “comprises”, “comprised” or “comprising” are used in this specification (including the provisional claims) they are to be interpreted as specifying the presence of the stated features, integers, steps or components, but not precluding the presence of one or more other features, integers, steps or components or group thereof.

Future patent applications may be filed on the basis of or claim priority from the present application. It is to be understood that the following claims are provided by way of example only, and are not intended to limit the scope of what may be claimed in future applications. Features may be added to or omitted from the provisional claims at a later date so as to further define or re-define the invention or inventions.

1. A device for providing respiratory support to a patient, the device comprising:
(a) a first gas flow path;
(b) a second gas flow path;
(c) a first gases port configured to receive gases from either of the first gas flow path and the second gas flow path; and
(d) a switching mechanism operable to switch flow to the first gases port between the first gas flow path and the second gas flow path;
wherein gases from the first gases port are provided to the patient for respiratory support.

2. The device according to claim 1, wherein operation of the switching mechanism to allow flow from one of the gas flow paths to the first gases port prevents flow from the other of the gas flow paths to the first gases port.

3. The device according to claim 1 or claim 2, wherein the first gases port is couplable with a first conduit providing gases to the patient through a second patient interface.

4. The device according to any one of the preceding claims, comprising a second gases port which is couplable with a second conduit configured to receive expired gases from the patient.

5. The device according to claim 4, further comprising first patient interface configured to receive expired gases from the patient for return to the device through the second conduit.

6. The device according to claim 5, wherein application of the first patient interface to the patient causes the switching mechanism to allow flow to the first gases port from the first gas flow path.

7. The device according to claim 5 or claim 6, wherein application of the first patient interface to the patient is determined by detecting an increase in one or more parameters of gases an expiratory gas flow path between the first patient interface and the device, the parameters selected from a group including, gas pressure, CO2, O2, flow rate, temperature and humidity.

8. The device according to claim 7, wherein the first patient interface comprises one or more sensors configured to determine when the first patient interface has been applied to the patient.

9. The device according to claim 8, wherein the one or more sensors of the first patient interface include one or more of an optical sensor, a proximity sensor and a thermal sensor.

10. The device according to any one of claims 5 to 9, wherein the first patient interface is a face mask.

11. The device according to claim 10, wherein the face mask comprises a sealing cuff having a sensor for determining pressure within the cuff, and wherein application of the face mask to the patient is determined by an increase in cuff pressure.

12. The device according to any one of claims 5 to 11, comprising an endotracheal tube (ETT) or laryngeal mask airway (LMA) couplable with the first and second conduits via a wye-piece connector, and functioning as both the first patient interface and the second patient interface.

13. The device according to claim 12, wherein the device is configurable for use as a ventilator.

14. The device according to any one of claims 5 to 13, wherein removal of the first patient interface from the patient causes the switching mechanism to allow flow to the first gases port from the second gas flow path.

15. The device according to any one of the preceding claims, wherein, when gases flow to the first gases port from the second gas flow path, the device is operable to provide gases to the patient through a second patient interface being a non-sealing patient interface.

16. The device according to claim 15, wherein the non-sealing patient interface is a nasal cannula.

17. The device according to any one of the preceding claims, wherein the switching mechanism comprises a switching element which, when the switching mechanism operates to allow flow from one of the gas flow paths to the first gases port, is configured to prevent flow from a flow source to the other gas flow path.

18. The device according to any one of the preceding claims, wherein the switching mechanism comprises one or more valves upstream of a junction between the first gases flow path and the second gases flow path which, when the switching mechanism operates to allow flow from one of the gas flow paths to the first gases port, prevent backflow in the other gas flow path.

19. The device according to any one of the preceding claims, wherein the switching mechanism is operatively coupled with one or more flow meters configured to stop a gases flow to the gas flow path that does not provide a flow of gas to the first gases port according to operation of the switching mechanism.

20. The device according to any one of the preceding claims, wherein the switching mechanism is operatively coupled with one or more flow meters operable to control a gases flow to the gas flow path providing a flow of gases to the first gases port according to operation of the switching mechanism.

21. The device according to any one of the preceding claims, wherein the switching mechanism is operatively coupled with one or more flow meters operable to control a gases flow to the gas flow path providing a flow of gases to the first gases port according to operation of the switching mechanism, wherein the one or more fixed flow meters control the gases flow at one or more pre-set gas flow rates.

22. The device according to any one of the preceding claims, comprising a controller in operable communication with the switching mechanism and operable to control the device to deliver respiratory support to the patient.

23. The device according to claim 22, wherein the controller receives inputs from one or more sensors for determining if the first patient interface has been applied to the patient.

24. The device according to claim 23, wherein:
upon determination that the first patient interface has been applied to the patient, the controller automatically controls the device to provide respiratory support in a first mode; and
upon determination that the first patient interface has been removed from the patient, the controller automatically controls the device to provide respiratory support in a second mode.

25. The device according to claim 23 or claim 24, wherein the controller receives inputs from one or more sensors in one or more locations selected from a group including:
in an inspiratory flow path providing gases to a patient;
in the device between the first gases port and a junction where returned gases in the device meet with fresh gas supply;
in an expiratory flow path returning gases from the patient to the device;
in the device between a second gases port receiving returned gases from the patient and the junction; and
in a gases flow downstream of a flow generator or flow mixer of the device.

26. The device according to claim 25, wherein the one or more sensors are a sensor type selected from a group comprising pressure sensors, flow rate sensors and O2 sensors.

27. The device according to any one of claims 23 to 26, wherein the controller receives inputs from one or more CO2 sensors in one or more locations selected from a group including:
an expiratory flow path returning gases from the patient to the device; and
in a device having a rebreathing circuit and CO2 absorber, in the device between a second gases port receiving returned gases from the patient and the CO2 absorber.

28. The device according to claim 26, wherein the controller determines the one or more locations containing a CO2 concentration higher than ambient air is associated with the patient interface that is applied to the patient.

29. The device according to any one of claims 24 to 25, wherein the controller receives inputs from multiple sensors to determine if the first patient interface has been applied to the patient to mitigate erroneous switching between the first mode and the second mode.

30. The device according to any one of claims 23 to 26, wherein the controller controls the device to provide respiratory support with a signature flow element, and wherein the controller looks for the signature flow element in returned gases to determine that the first patient interface has been applied to the patient, and optionally wherein the signature flow element comprises an oscillation in one or more of frequency, amplitude and profile of one or more of pressure, flow rate and O2 concentration of the gases provided to the patient.

31. The device according to any one of claims 24 to 29, wherein the controller receives sensor signals from one or more sensors comprising one or more pressure and/or flow rate and/or gas concentration sensors in an inspiratory flow path and/or an expiratory flow path to control switching from the second mode to the first mode, and optionally wherein the same or different one or more sensor signals may be received by the controller to control switching from the first mode to the second mode.

32. The device according to claim 30, wherein the controller is configurable to require more sensor conditions to be met before switching from the first mode to the second mode, or vice versa.

33. The device according to any one of claims 23 to 32, wherein the controller controls the device to provide a residual flow of gas in one or both of the first gas flow path and the second gas flow path for continuous or regular monitoring of gas, and optionally wherein the residual flow is from about 0.5 L/min to about 5 L/min.

34. The device according to any one of the preceding claims, comprising one or more sensors sensing one or more characteristics of gas selected from a group including: pressure, flow rate and gas species concentration.

35. The device according to any one of claims 24 to 34, wherein the controller applies a timing control, wherein upon detection of a condition that should trigger a mode switching, the controller applies a delay before controlling the switching elements to cause the mode switch.

36. The device according to claim 35, wherein the controller controls a user interface to provide one or more of an audible and visible countdown indicator of when the controller will switch control of the device between the first mode and the second mode, and optionally wherein the controller is configured to abandon an automated switch between modes upon receipt of a user input to cancel during the delay.

37. The device according to any one of the preceding claims, wherein the controller controls a user interface providing one or both of an audible and visible mode indication representing a current operating mode of the device and optionally, wherein the user interface comprises one or more audible and/or visible output elements located on, at or near a gas conduit or patient interface and providing the mode indication.

38. The device according to any one of claims 22 to 37, comprising a user interface in operable communication with the controller and configured to receive input from a user corresponding to one or more parameters of the respiratory support to be provided to the patient.

39. The device according to claim 38, wherein the parameters comprise one or more parameters for a rebreathing mode of respiratory support and/or a high flow mode of respiratory support.

40. The device according to claim 38 or claim 39, wherein the parameters comprise one or more of flow rate, composition, pressure, temperature and humidity of gases to be provided to the patient.

41. The device according to any one of claims 22 to 40, wherein the controller is configured to receive a user input causing the switching mechanism to switch flow to the first gases port between the first gas flow path and the second gas flow path.

42. The device according to claim 41, wherein the user input is received from one or more of:
(a) a touch screen display;
(b) a wired or wirelessly coupled remote unit;
(c) a foot operated pedal or switch; and
(d) a physical switch provided on the device.

43. The device according to any one of claims 5 to 42 when appended to claim 4, wherein the first gas flow path comprises a rebreathing gas flow path which receives and processes expired gases returned from the patient.

44. The device according to claim 43 wherein the device is operable to provide one or both of anaesthesia and ventilatory respiratory support via the rebreathing gas flow path.

45. The device according to any one of the preceding claims, wherein the second gas flow path comprises a high flow gas flow path.

46. The device according to any one of the preceding claims, wherein the first gases port is a common gases outlet port which is couplable with a first conduit for delivering gases to a patient from either the first gas flow path or the second gas flow path.

47. The device according to any one of the preceding claims, comprising a housing in which the first gas flow path and the second gas flow path are provided, the housing providing a first gases coupling defining the first gases port with which a first conduit is couplable.

48. The device according to any one of the preceding claims, comprising a humidifier configured to condition gases to a pre-determined temperature and/or humidity before delivery to the patient via one or both of the first and second gas flow paths.

49. The device according to any one of the preceding claims, operable to provide a flow of gases in the second gas flow path at a flow rate that is selectable from an available range of about 20 L/min to about 100 L/min.

50. The device according to any one of claims 1 to 48, operable to provide a flow of gases in the second flow path at a flow rate that is selectable from a plurality of available fixed flow rates including at least 0 L/min, 40 L/min and 70 L/min.

51. The device according to claim 49 or claim 50, comprising a flow source configured to generate the flow of gases in the second gas flow path.

52. The device according to any one of claims 5 to 51 when appended to claim 4, comprising a CO2 absorber configured to treat expired gases returned from the patient in the first gas flow path.

53. The device according to any one of the preceding claims, comprising one or more of the following features in the first gas flow path:
(a) a pressure limiting valve configured to maintain substantially stable pressure in the first gas flow path;
(b) a variable volume for displacement of gases in the first gas flow path;
(c) a replenishing gas flow for replenishing anaesthetic gas delivered to the patient in the first gas flow path;
(d) a gas mixer;
(e) a vaporizer for vaporizing volatile anaesthetic agents into gas in the first gas flow path; and
(f) a flow rate limiter configured to limit flow in the first gas flow path to about 15 L/min.

54. The device according to any one of the preceding claims, comprising one or more gases supply ports configured to receive a supply of one or both of:
(a) a breathing gas for delivery to the patient by the first or the second gas flow path; and
(b) an anaesthetic gas for delivery to the patient by the first gas flow path.

55. The device according to any one of claims 5 to 54 when appended to claim 4, configured for operation:
(a) in a rebreathing mode wherein gases flow from the first gas flow path to the first gases port and are provided to the patient, and expired patient gases are returned to the device via the second gases port; and
(b) a high flow mode wherein gases flow from the second gas flow path to the first gases port and are provided to the patient without expired patient gases being returned to the device.

56. The device according to claim 55, wherein the rebreathing mode comprises an anaesthetic rebreathing mode for provision of anaesthesia to the patient.

57. The device according to claim 55 or claim 56, configured for use in a ventilation rebreathing mode wherein gases flow from the first gas flow path to the first gases port and are provided to the patient through an ETT or LMA from which expired patient gases are returned to the device via the second gases port.

58. A device for providing respiratory support to a patient, the device comprising:
a first gas flow path;
a second gas flow path;
a first gases port configured to receive gases from either of the first gas flow path and the second gas flow path; and
a switching mechanism operable to switch flow to the first gases port between the first gas flow path and the second gas flow path;
wherein gases from the first gases port are provided to the patient for respiratory support; and
wherein the device comprises a controller configured to receive inputs from one or more sensors for determining if a condition exists to trigger switching the flow to the first gases port between the first gas flow path and the second gas flow path, wherein the one or more sensors are provided in one or more locations selected from a group including:
in an inspiratory flow path providing gases to a patient;
in the device between the first gases port and a junction where returned gases in the device meet with fresh gas supply;
in an expiratory flow path returning gases from the patient to the device;
in the device between a second gases port receiving returned gases from the patient and the junction; and
in a gases flow downstream of a flow generator or flow mixer of the device.

59. The device according to claim 58, wherein the device comprises a second gases port configured to receive gases returned from the patient, and the condition comprises the controller determining if a sealing patient interface in fluid communication with second gases port is, or is not, applied to the patient.

60. A device for providing respiratory support to a patient, the device comprising:
a first gas flow path;
a second gas flow path;
a first gases port configured to receive gases from either of the first gas flow path and the second gas flow path; and
a switching mechanism operable to switch flow to the first gases port between the first gas flow path and the second gas flow path;
wherein gases from the first gases port are provided to the patient for respiratory support; and
wherein the device comprises a controller configured to control the device to provide respiratory support with a signature flow element, and wherein the controller looks for the signature flow element in gases returned from the patient to the device to determine if a patient interface has been applied to the patient.

61. The device according to claim 60, the signature flow element comprises an oscillation in one or more of frequency, amplitude and profile of one or more of pressure, flow rate and O2 concentration of the gases provided to the patient.
